(19) United States
(12) Patent Application Publication
(10) Pub. No.: US 2010/0256229 A1
(43) Pub. Date: Oct. 7, 2010

(54) COMPOSITIONS OF CHOLINESTERASE INHIBITORS

(75) Inventors: Shoucheng Du, Pittsburgh, PA (US); Marc W. Andersen, Raleigh, NC (US); Daniel Coughlin, Hackettstown, NJ (US); Alexander Kolchinski, Winchester, MA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Colucid Pharmaceuticals, Inc., Durham, NC (US)

(21) Appl. No.: 12/753,567

(22) Filed: Apr. 2, 2010

Related U.S. Application Data
(60) Provisional application No. 61/166,143, filed on Apr. 2, 2009.

(51) Int. Cl. A61K 31/27; C07C 271/44; A61P 27/06; A61P 25/16; A61P 25/28; A61P 1/00; A61P 9/10; A61P 25/34

(52) U.S. Cl. 514/487; 560/32

(57) ABSTRACT

The present invention is directed to compositions, methods of use, and processes for the synthesis related to 3-((S)-1-(dimethylamino)ethyl)phenyl methyl-((R)-1-phenylpropan-2 yl)carbamate, and its pharmaceutically acceptable salt forms, including the hydrogen fumarate salt. The present invention also relates to a novel form polymorph of 3-((S)-1-(dimethylamino)ethyl)phenylmethyl-((R)-1-phenylpropan-2 yl)carbamate, characterized by a unique X-ray diffraction pattern and Differential Scanning Calorimetry profile, as well a unique crystalline structure.

### RELATED APPLICATION

This application claims priority under 35 U.S.C. §119 to U.S. Ser. No. 61/166,143, filed Apr. 2, 2009, the contents of which are incorporated herein.

### FIELD OF THE INVENTION

The present invention is directed to compositions, methods of use and processes for the synthesis of substantially pure cholinesterase inhibitors, specifically 3-((S)-1-(dimethylamino)ethyl)phenyl methyl-((R)-1-phenylpropan-2-yl)carbamate, and its pharmaceutically acceptable salt forms, including the hydrogen fumarate salt. The invention is also directed to certain polymorphs and methods of using such polymorphs.

### BACKGROUND OF THE INVENTION

Cholinesterase inhibitors (ChEIs) of the carbamate type (“stigmines”) are known. Towards the end of the nineteenth century, physostigmine found medicinal use in the treatment of glaucoma. More recently, physostigmine has been used for treatment of myasthenia gravis and Alzheimer disease. However, the severity of the side effects associated with high doses of physostigmine has spurred the search for other carbamate cholinesterase inhibitors that are safer and better tolerated. Examples of such stigmines are the approved AD drug rivastigmine (marketed as Exelon®) and the experimental AD drug phenserine. Although these second generation cholinesterase inhibitors are better tolerated than physostigmine, their application is still hampered by limited efficacy and a narrow therapeutic window. Shown below are representative examples of carbamate cholinesterases (“stigmines”):

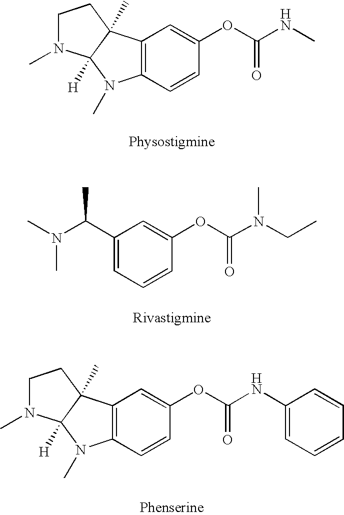

Stigmines inhibit AChE by transferring their carbamoyl group to a serine residue in the active site (semi-irreversible inhibition, see the schematic mechanism shown below). The covalently bound carbamate is slowly hydrolyzed to reconstitute the active enzyme. During this process, a carbamic acid is released that in turn dissociates into carbon dioxide and an amine. In known stigmines, this amine is a small molecule that is considered pharmacologically inactive. We hypothesized that this mechanism could be leveraged to release a biologically active amine during the process of carbamic acid dissociation. Thus, these stigmines could provide both cholinesterase inhibition and actions at additional relevant targets in a single molecule, potentially leading to increased efficacy and tolerability compared to known cholinesterase inhibitors. Shown below is a schematic mechanism of action for cholinesterase inhibition by carbamates:

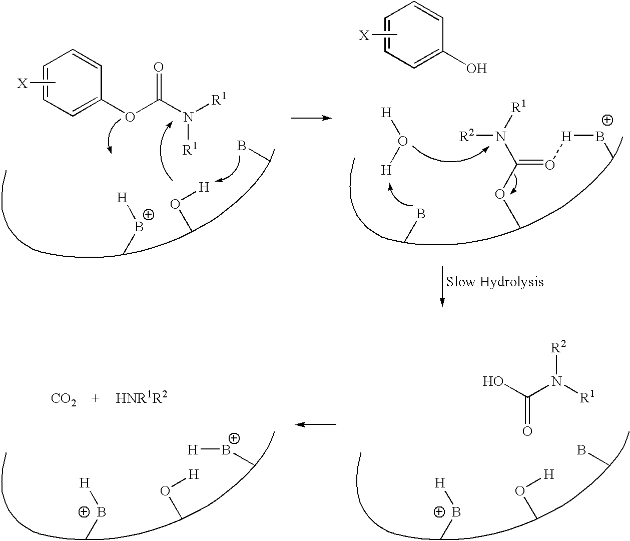

Recently, the concept of providing ChEIs with additional pharmacology has received increased attention.

### SUMMARY OF THE INVENTION

The present invention relates to a hydrogen fumarate salt of compound 3d:

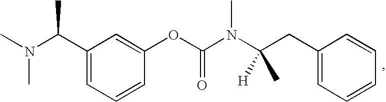

characterized by an x-ray diffraction pattern substantially similar to that set forth in FIG. 16.

The present invention relates to a pharmaceutical composition comprising the hydrogen fumarate salt of compound 3d:

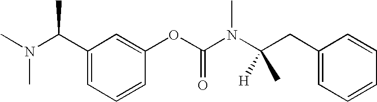

and a pharmaceutically acceptable carrier or excipient.

In one aspect of the invention, the compound has a purity of greater than 98.0% as determined by LCMS. In one aspect of the invention, the compound contains less than 2% impurity. In one aspect of the invention, the compound contains less than 2% d-methamphetamine.

The present invention relates to a method of treating or preventing a nervous system condition, cholinergic deficiency or glaucoma in an individual by administering a composition of the invention. The present invention relates to the use of a composition of the invention in the manufacture of a medicament for treating or preventing an individual having a nervous system condition, cholinergic deficiency or glaucoma. In one aspect, the nervous system condition is selected from a central nervous system condition, a peripheral nervous system condition, and autonomic nervous system condition. In one aspect, the central nervous system condition is selected from Parkinson's disease, memory impairment, and cognitive impairment.

In one aspect, the memory impairment is selected from Alzheimer's disease, age associated memory loss, impairment in memory consolidation, impairment in short term memory, mild cognitive impairment, and multiple sclerosis.

The present invention relates to a method of treating or preventing a condition associated with acetylcholinesterase activity in an individual by administering a composition of the invention, wherein the condition is selected from delayed gastric emptying, attention deficit hyperactivity disorder (ADHD), phobia, stroke, multiple sclerosis, sleep disorder, psychiatric disorder, pain, anticholinergic drug overdose, tobacco dependence, and spasticity. The present invention relates to the use of a composition of the invention in the manufacture of a medicament far treating or preventing an individual having a condition associated with acetylcholinesterase activity, wherein the condition is selected from delayed gastric emptying, attention deficit hyperactivity disorder (ADHD), phobia, stroke, multiple sclerosis, sleep disorder, psychiatric disorder, pain, anticholinergic drug overdose, tobacco dependence, and spasticity.

The present invention relates to a method of promoting wakefulness in an individual by administering a composition of the invention. The present invention relates to the use of a composition of the invention in the manufacture of a medicament for promoting wakefulness in an individual. In one aspect, the individual suffers from a disorder or condition selected from wakefulness disorders, hypersomnia, sleep apnea, sleep disorders of central origin, fatigue, excessive daytime sleepiness associated with narcolepsy, fatigue and excessive sleepiness associated with a depressive disorder or with antidepressant therapy.

The present invention relates to methods, wherein the individual is a human.

The present invention relates to a process for preparing 3-((S)-1-(dimethylamino)ethyl)phenyl methyl-((R)-1-phenylpropan-2 yl)carbamate hydrogen fumarate comprising the steps of: (1) reacting L-methamphetamine with carbonyldiimidazole to form (R)—N-Methyl-N-(1-phenylpropan-2-yl)-1H-imidazole-1-carboxamide; (2) reacting (R)—N-Methyl-N-(1-phenylpropan-2-yl)-1H-imidazole-1-carboxamide with (S)-rivalphenol to form 3-((S)-1-(dimethylamino)ethyl)phenyl methyl-((R)-1-phenylpropan-2 yl)carbamate; and (3) contacting 3-((S)-1-(dimethylamino)ethyl)phenyl methyl-((R)-1-phenylpropan-2 yl)carbamate with fumaric acid to yield 3-((S)-1-(dimethylamino)ethyl)phenyl methyl-((R)-1-phenylpropan-2 yl)carbamate hydrogen fumarate.

The present invention relates to a compound having the structure

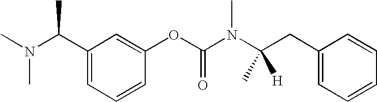

or a pharmaceutically acceptable salt thereof.

### BRIEF DESCRIPTION OF THE DRAWINGS

FIG. 1 is a table showing a comparison of three salt forms of compound 3d.

FIG. 2 is two microscopic views showing hygroscopicity testing of the hydrogen sulfate salt of compound 3d which deliquesces after 3 days at 40° C./75 RH. The views A-1 and A-2 show the appearance after three and seven days at 75% RH, respectively.

FIG. 3 is two microscopic views showing hygroscopicity testing of the hydrogen fumarate salt of compound 3d which is a solid after 7 days at 40° C./75 RH. Analysis of compound 3d hydrogen fumarate by microscopy indicates that the salt remains crystalline with traces of amorphous material after 7 days at 75% relative humidity.

FIG. 4 is photograph showing a gelatin capsule, size #000 filled with 200 mg of compound 3d hydrogen fumarate. The API fit in to size #000 with minimal effort and manual packing. This indicates that 200 mg of API can fit into the next smallest capsule size #00.

FIG. 5 is a HPLC trace showing the results of chiral HPLC separation for all possible isomers of compound 3d. No isomer impurities are observed in batches of compound 3d.

FIG. 6 is an HPLC trace showing the results of forced degradation of compound 3d in solution under an ultraviolet lamp for 2 hours 45 min.

FIG. 7 is a table showing the results of a salt selection study of 15 salts of compound 3d using IPA as a solvent.

FIG. 8 is a table summarizing melting point data for the hydrogen succinate salt of compound 3d.

FIG. 9 is a table showing a comparison of solid state properties of samples of hydrogen fumarate and hydrogen sulfate samples of compound 3d.

FIG. 10 is a table showing a comparison of hygroscopicity of the hydrogen fumarate and hydrogen sulfate salt forms of compound 3d.

FIG. 11 is a graph showing the sorption isotherm of compound 3d. Compound 3d does not absorb much water during absorption phase; maximum moisture uptake is about 1% (at 75 RH). Adsorption appears reversible.

FIG. 12 is a table showing the solid state stability of the hydrogen fumarate salt of compound 3d. Samples of compound 3d hydrogen fumarate were stable to heat (40° C.) for 7 days and to UV (220 W-hr/m²) and visible light for 7.3×10⁶ lux hours.

FIG. 13 is two microscopic views of the hydrogen fumarate salt of compound 3d under mechanical stress. Micronizing a sample of the fumarate salt of compound 3d for 20 minutes at 30 Hz resulted in “gumming” after standing for 20 minutes.

FIG. 14 is a table showing the chemical stability of the hydrogen fumarate salt of compound 3d at 50 min, 2 hour, 24 hour, and 48 hour time points.

FIG. 15 is a table showing the chemical stability of the hydrogen fumarate salt of compound 3d at 24 hour, 48 hour, 5 day and 7 day time points. The hydrogen fumarate salt of compound 3d was stable in water for up to 7 days at 40° C.

FIG. 16 is an X-ray diffractogram for the hydrogen fumarate salt of compound 3d.

FIG. 17 is a DSC thermogram for the hydrogen fumarate salt of compound 3d.

FIG. 18 is a X-ray diffractogram for the hydrogen succinate salt of compound 3d.

FIG. 19 is a DSC thermogram for the hydrogen succinate salt of compound 3d.

FIG. 20 is a X-ray diffractogram for the hydrogen sulfate salt of compound 3d.

FIG. 21 is a DSC thermogram for the hydrogen sulfate salt of compound 3d.

FIG. 22 is a ¹HNMR spectrum of the free base of compound 3d.

FIG. 23 is a ¹HNMR spectrum of the hydrogen fumarate salt of compound 3d.

FIG. 24 is a ¹HNMR spectrum of the hydrogen sulfate salt of compound 3d.

FIG. 25 is a ¹HNMR spectrum of the hydrogen succinate salt of compound 3d.

FIG. 26 is an ¹HNMR spectrum of a urea intermediate isolated during the preparation of compound 3d.

FIG. 27 is a bar graph that shows the step-through latencies in a rat passive avoidance model). Rats received a dose of 0.2 mg/kg scopolamine s.c. 40 minutes before training and doses of 0, 8, 12, 16, or 24 mg of 4a i.p. 30 minutes before training (Example 6).

FIG. 28 is a graph that shows total minutes of sleep loss versus dose of compounds of the invention, modafinil, d-amphetamine, and rivastigmine (Example 12).

FIG. 29 is a graph that shows that rebound hypersomnolence is not observed following administration of Compound B (Example 12).

FIG. 30 is a graph that shows the Compound B did not cause an increase in body temperature (Example 12).

FIG. 31 is a graph that shows that Compound B did not cause hyperactivity indicating an absence of stimulant activity (Example 12).

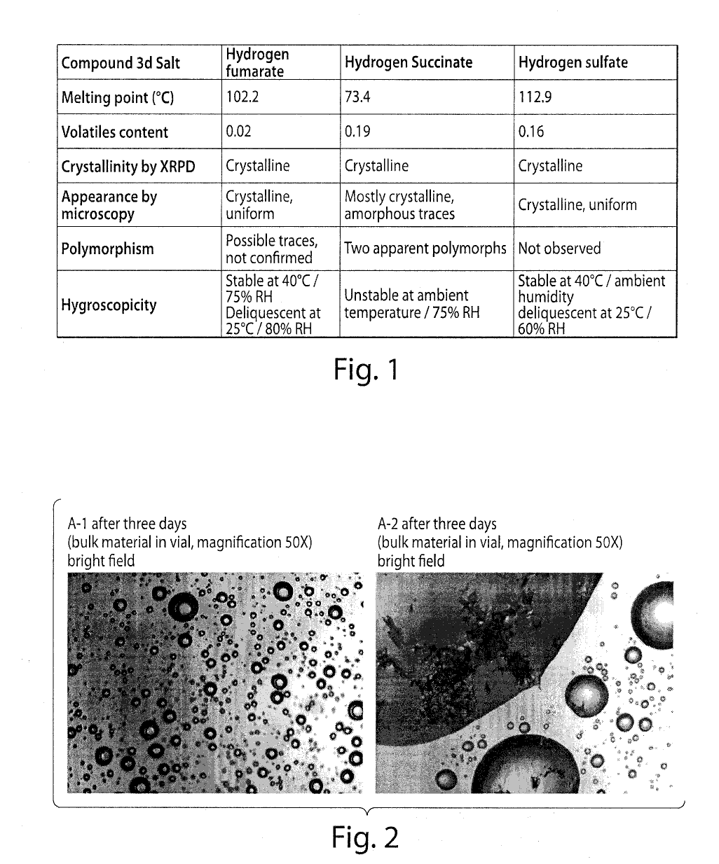

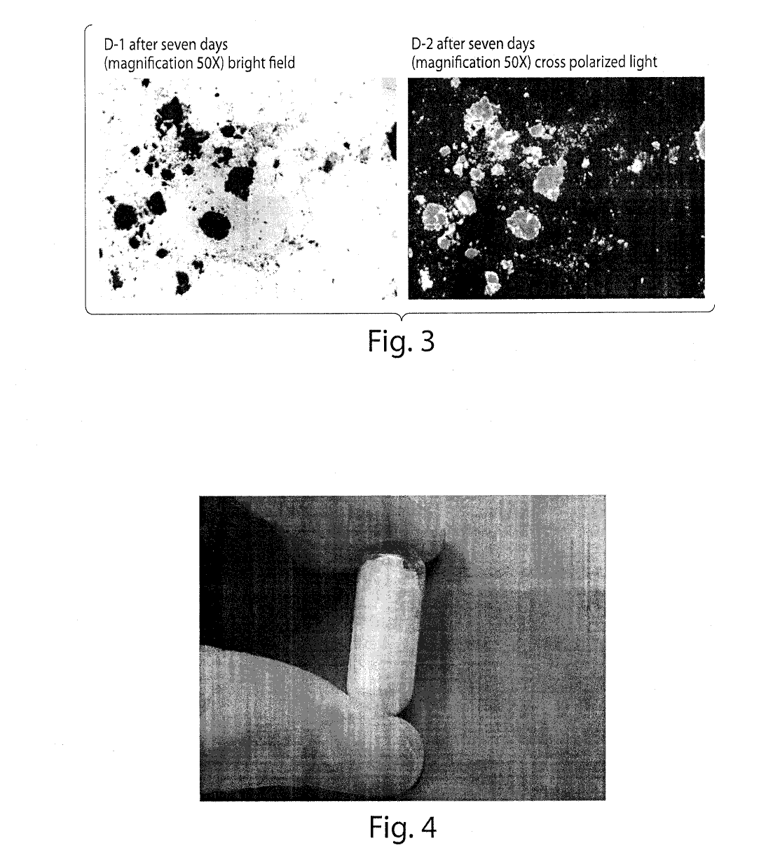

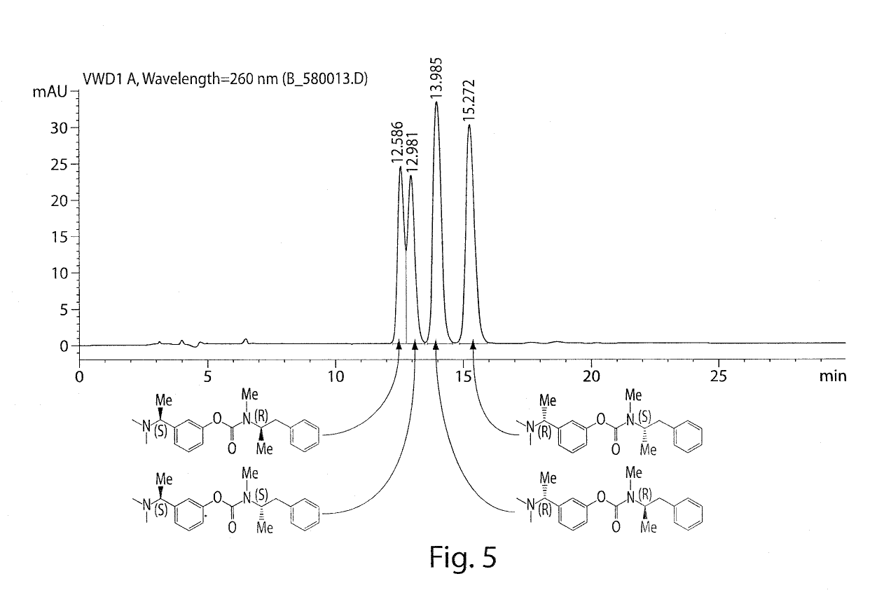

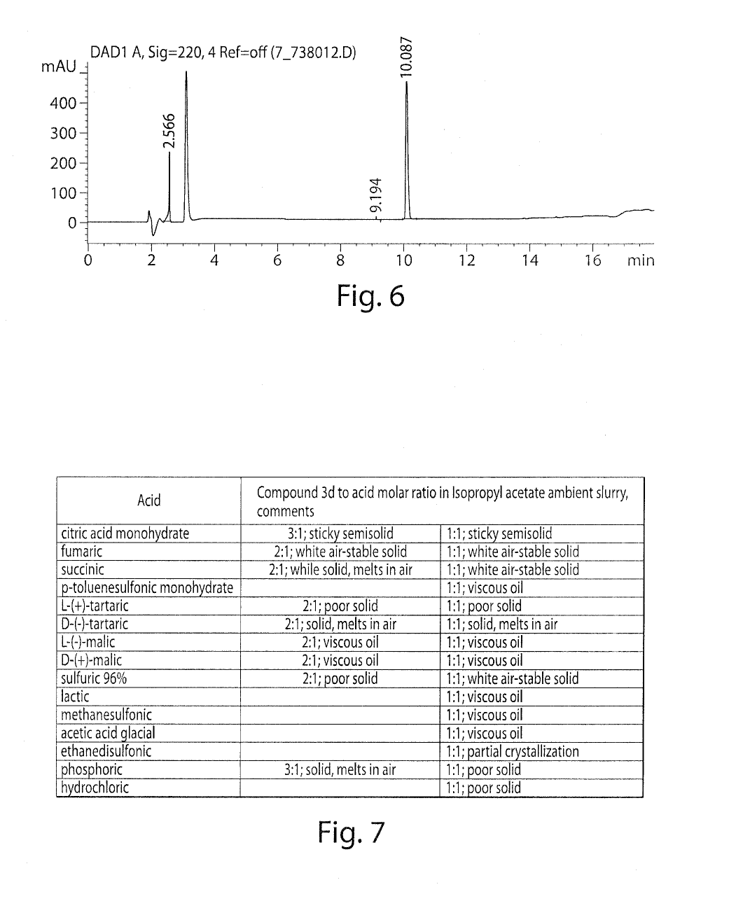

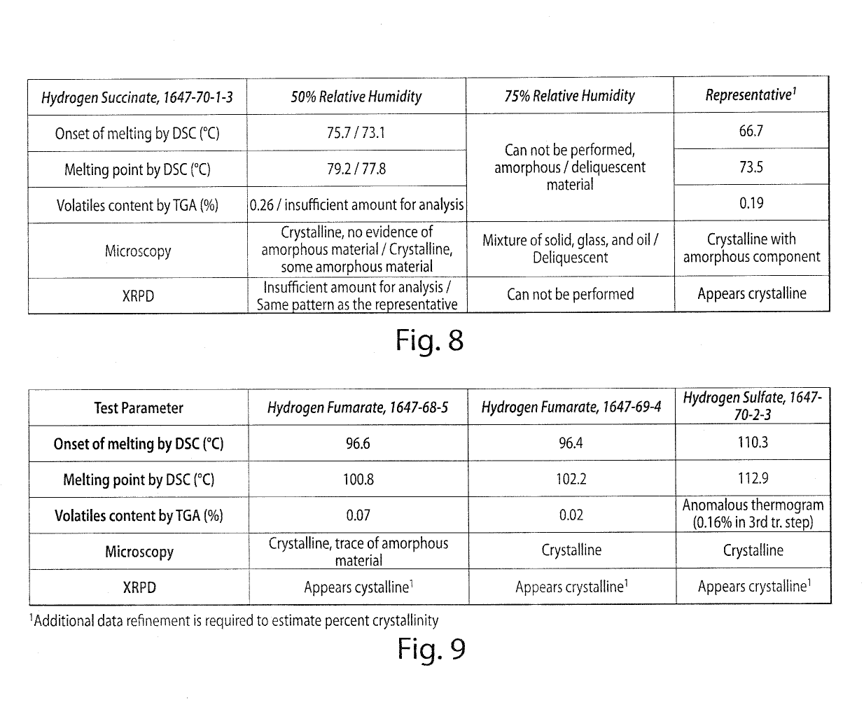

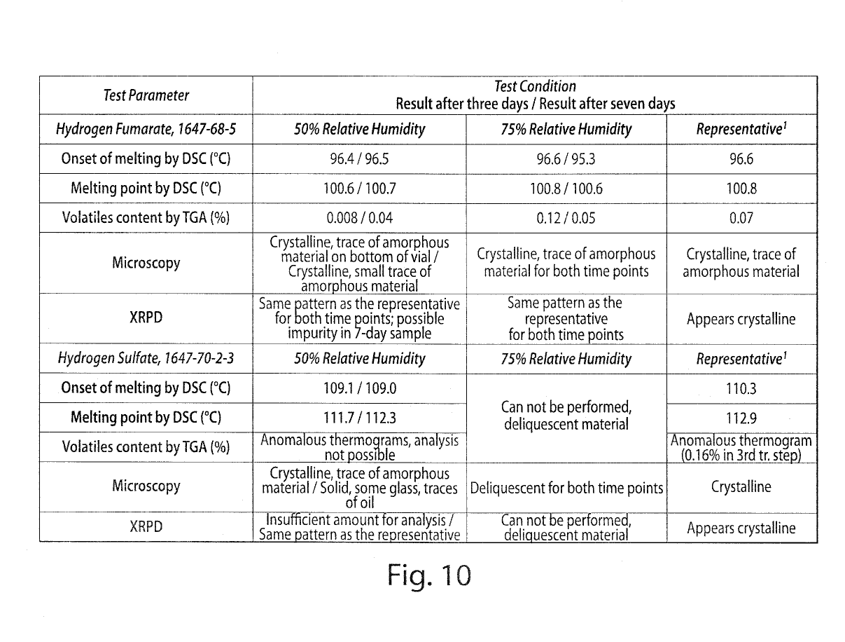

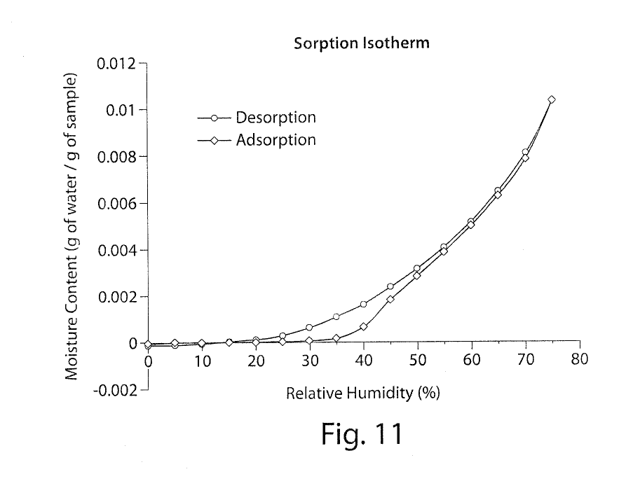

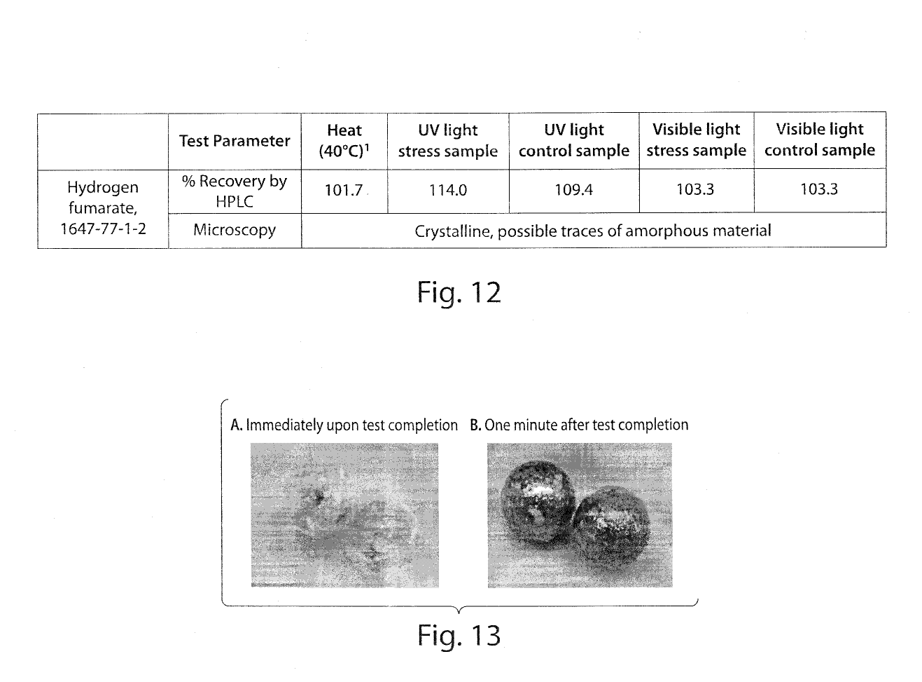

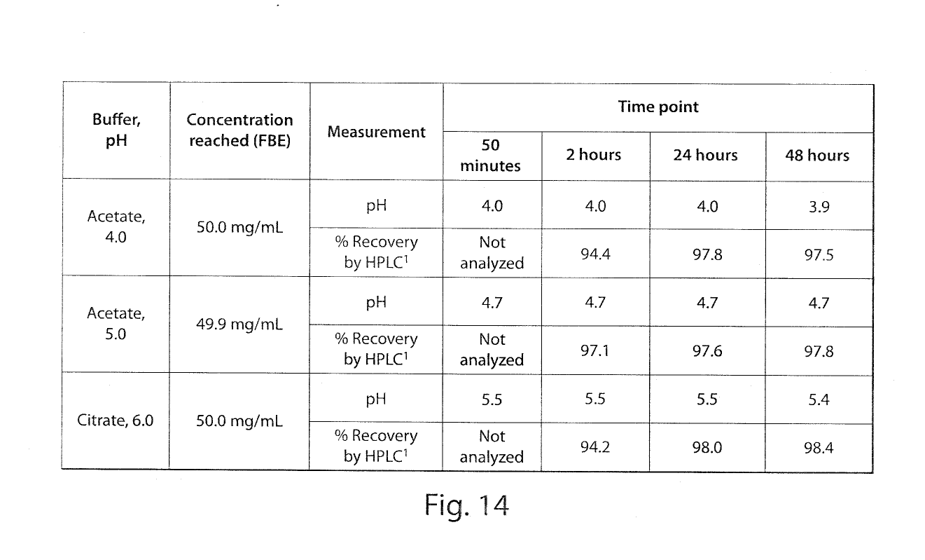

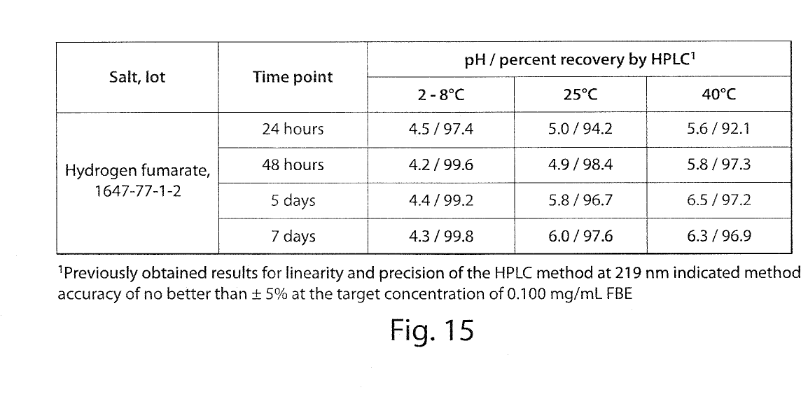

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The present invention is directed to a composition comprising a pharmaceutically acceptable salt of compound 3d:

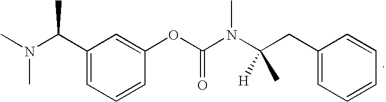

In one aspect, the composition of the invention is the hydrogen fumarate salt of compound 3d. In one aspect, the composition of the invention is the hydrogen succinate salt of compound 3d. In one aspect, the composition of the invention is the hydrogen sulfate salt of compound 3d.

In one aspect, the composition of the invention comprises compound 3d having a purity greater than 98.0% as determined by LCMS. In one aspect, the composition of the invention comprises compound 3d having a purity of greater than 99.0% as determined by LCMS. In one aspect, the composition of the invention comprises compound 3d having a purity of greater than 99.5% as determined by LCMS. In one aspect, the composition of the invention comprises a salt of compound 3d having a purity of greater than 99.7% as determined by LCMS.

In one aspect, the composition of the invention comprises a salt of compound 3d having a purity of greater than 98.0% as determined by LCMS. In one aspect, the composition of the invention comprises a salt of compound 3d having a purity of greater than 99.0% as determined by LCMS. In one aspect, the composition of the invention comprises a salt of compound 3d having a purity of greater than 99.5% as determined by LCMS. In one aspect, the composition of the invention comprises a salt of compound 3d having a purity of greater than 99.7% as determined by LCMS.

In one aspect, the composition of the invention contains less than 2% impurity. In one aspect, the composition of the invention contains less than 1% impurity. In one aspect, the composition of the invention contains less than 0.5% impurity. In one aspect, the composition of the invention contains less than 0.1% impurity. In one aspect, the composition of the invention contains 0% impurity.

In one aspect, the composition contains less than 2%, less than 1%, less than 0.5%, less than 0.1%, or 0% of an impurity such as unreacted starting matter or a by-product formed during the procedure to make the compound. Examples of impurities include activated urea:

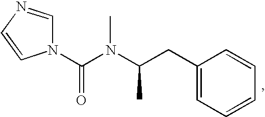

rivaphenol, L-methamphetamine, D-methamphetamine, and symmetrical urea

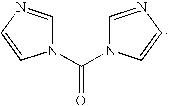

The present invention is directed to a composition of the invention further comprising a pharmaceutically acceptable carrier or excipient.

The present invention is directed to certain polymorphs of 3-((S)-1-(dimethylamino)ethyl)phenyl methyl-((R)-1-phenylpropan-2 yl)carbamate, including polymorphs of its salt forms characterized by X-ray diffraction pattern, DSC thermogram and/or crystal structure, processes of making these polymorphs, pharmaceutical compositions comprising these polymorphs, and methods of treating or preventing a nervous system condition, cholinergic deficiency, and glaucoma; methods of increasing acetylcholine; methods of treating or preventing a condition associated with acetylcholinesterase activity; and methods of promoting wakefulness.

The present invention is directed to a hydrogen fumarate salt of compound 3d:

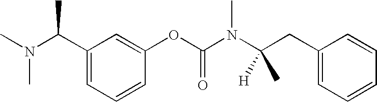

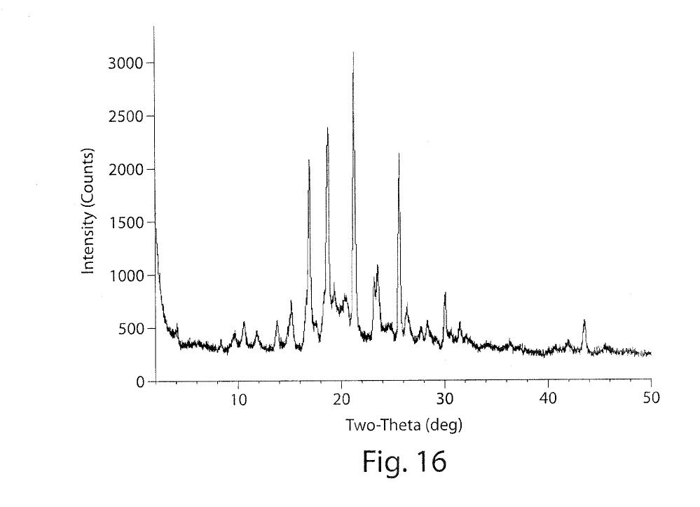

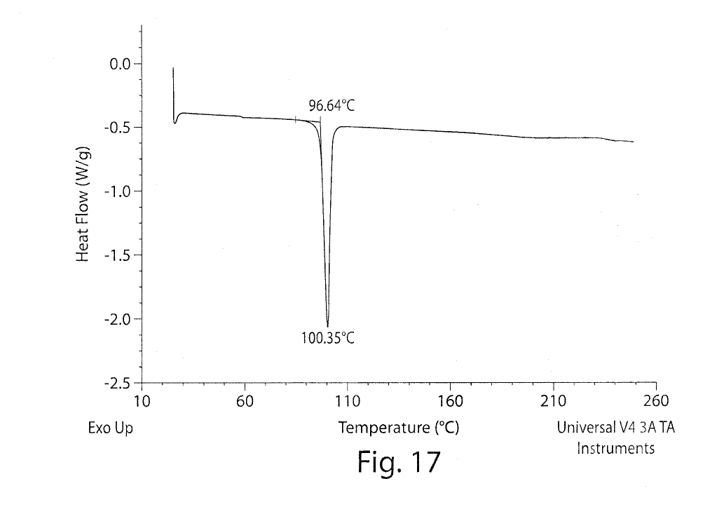

characterized by an X-ray diffraction pattern substantially similar to that set forth in FIG. 16. The hydrogen fumarate salt of compound 3d is characterized by an X-ray diffraction pattern including characteristic peaks at about 15.3, 17.0, 18.8, 21.3, 23.2, 23.6, 25.7, and 30.1 degrees 2-theta. The hydrogen fumarate salt of compound 3d is further characterized by an X-ray diffraction pattern including characteristic peaks at about 9.8, 10.6, 11.9, 13.8, 15.3, 17.0, 18.8, 21.3, 23.2, 23.6, 25.7, 26.4, 28.3, and 30.1 degrees two-theta.

The present invention is also directed to a hydrogen fumarate salt of compound 3d characterized by a Differential Scanning Calorimetry (DSC) thermogram having a single maximum value at about 100.35, as measured by a DSC Q100 V9.8 Build 96 instrument.

A further embodiment of the present invention is a hydrogen fumarate salt of compound 3d characterized by an X-ray diffraction pattern substantially similar to that set forth in FIG. 16 and further characterized by a Differential Scanning Calorimetry (DSC) thermogram having a single maximum value at about 100.35, as measured by a DSC Q100 V9.8 Build 96 instrument.

Another embodiment of the present invention is a hydrogen fumarate salt of compound 3d characterized by an X-ray diffraction pattern including characteristic peaks at about 15.3, 17.0, 18.8, 21.3, 23.2, 23.6, 25.7, and 30.1 degrees 2-theta and further characterized by a Differential Scanning Calorimetry (DSC) thermogram having a single maximum value at about 100.35, as measured by a DSC Q100 V9.8 Build 96 instrument.

Yet another embodiment of the present invention is a hydrogen fumarate salt of compound 3d characterized by an X-ray diffraction pattern including characteristic peaks at about 9.8, 10.6, 11.9, 13.8, 15.3, 17.0, 18.8, 21.3, 23.2, 23.6, 25.7, 26.4, 28.3, and 30.1 degrees two-theta and further characterized by a Differential Scanning Calorimetry (DSC) thermogram having a single maximum value at about 100.35, as measured by a DSC Q100 V9.8 Build 96 instrument.

The present invention is directed to a hydrogen fumarate salt of compound 3d produced by a process comprising the steps of:

(1) reacting L-methamphetamine with carbonyldiimidazole to form (R)—N-Methyl-N-(1-phenylpropan-2-yl)-1H-imidazole-1-carboxamide;

(2) reacting (R)—N-Methyl-N-(1-phenylpropan-2-yl)-1H-imidazole-1-carboxamide with (S)-rivalphenol to form 3-((S)-1-(dimethylamino)ethyl)phenyl methyl-((R)-1-phenylpropan-2 yl)carbamate; and

(3) contacting 3-((S)-1-(dimethylamino)ethyl)phenyl methyl-((R)-1-phenylpropan-2 yl)carbamate with fumaric acid to yield 3-((S)-1-(dimethylamino)ethyl)phenyl methyl-((R)-1-phenylpropan-2 yl)carbamate hydrogen fumarate. A further embodiment of the invention is a hydrogen fumarate salt of compound 3d produced by the aforementioned process and further characterized by an X-ray diffraction pattern substantially similar to that set forth in FIG. 16. Another embodiment of this invention is a hydrogen fumarate salt of compound 3d produced by the aforementioned process and further characterized by an X-ray diffraction patter including characteristic peaks at about 15.3, 17.0, 18.8, 21.3, 23.2, 23.6, 25.7, and 30.1 degrees two-theta. Yet another embodiment of the invention is a hydrogen fumarate salt produced by the aforementioned process and further characterized by an X-ray diffraction pattern including characteristic peaks at about 9.8, 10.6, 11.9, 13.8, 15.3, 17.0, 18.8, 21.3, 23.2, 23.6, 25.7, 26.4, 28.3, and 30.1 degrees two-theta. Another embodiment of the invention is a hydrogen fumarate salt of compound 3d produced by the aforementioned process and further characterized by a DSC thermogram having a single maximum value at about 100.35, as measured by a DSC Q100 V9.8 Build 96 instrument.

A further embodiment of the invention is a hydrogen fumarate salt produced by the aforementioned process, further characterized by an X-ray diffraction pattern substantially similar to the set forth in FIG. 16 and by a DSC thermogram having a single maximum value at about 100.35, as measured by a DSC Q100 V9.8 Build 96 instrument.

Another embodiment of the invention is a hydrogen fumarate salt produced by the aforementioned process, further characterized by an X-ray diffraction pattern including characteristic peaks at about 15.3, 17.0, 18.8, 21.3, 23.2, 23.6, 25.7, and 30.1 degrees two-theta and by a DSC thermogram having a single maximum value at about 100.35, as measured by a DSC Q100 V9.8 Build 96 instrument.

Yet another embodiment of the invention is a hydrogen fumarate salt produced by the aforementioned process, further characterized by an X-ray diffraction pattern including characteristic peaks at about 9.8, 10.6, 11.9, 13.8, 15.3, 17.0, 18.8, 21.3, 23.2, 23.6, 25.7, 26.4, 28.3, and 30.1 degrees two-theta and by a DSC thermogram having a single maximum value at about 100.35, as measured by a DSC Q100 V9.8 Build 96 instrument.

The present invention is also directed at a hydrogen fumarate salt of compound 3d produced by a process comprising the step of recrystallizing a crude preparation of the hydrogen fumarate salt of compound 3d from an organic solvent or a mixture of an organic solvent and water. In particular embodiment, the hydrogen fumarate salt of compound 3d is produced by a process comprising the step of recrystallizing a crude preparation of the compound 3d from an organic solvent. The organic solvent may be an acetate such as isopropyl acetate (IPA). In another embodiment, the hydrogen fumarate salt of compound 3d is produced by a process comprising the step of recrystallizing a crude preparation of the compound 3d from a mixture of isopropyl acetate and acetone.

The present invention is directed to a hydrogen sulfate salt of compound 3d:

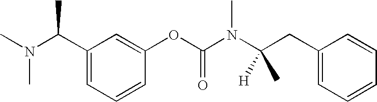

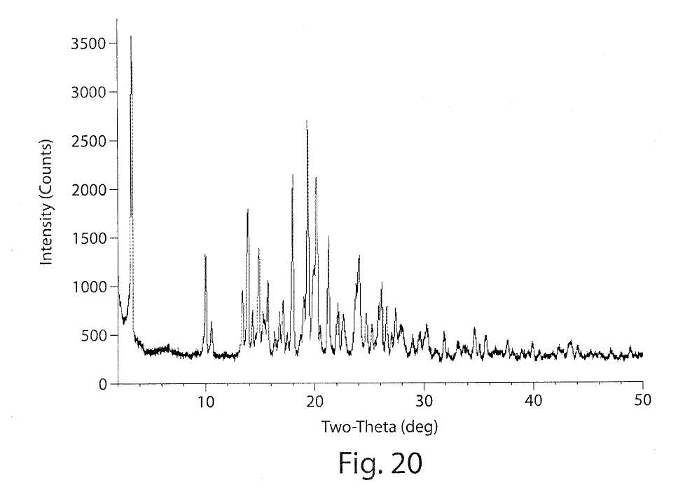

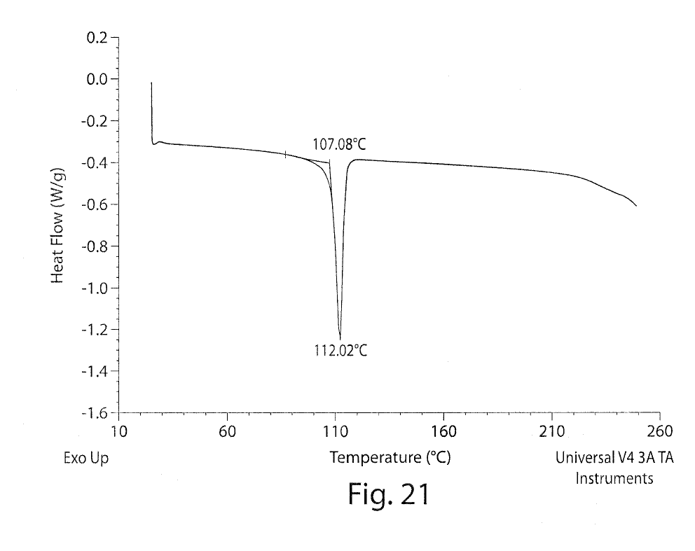

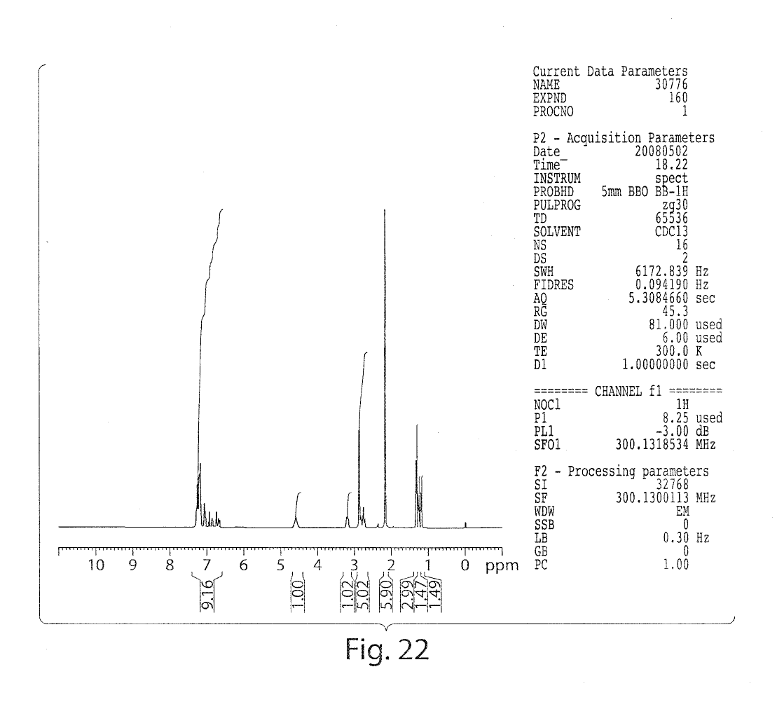

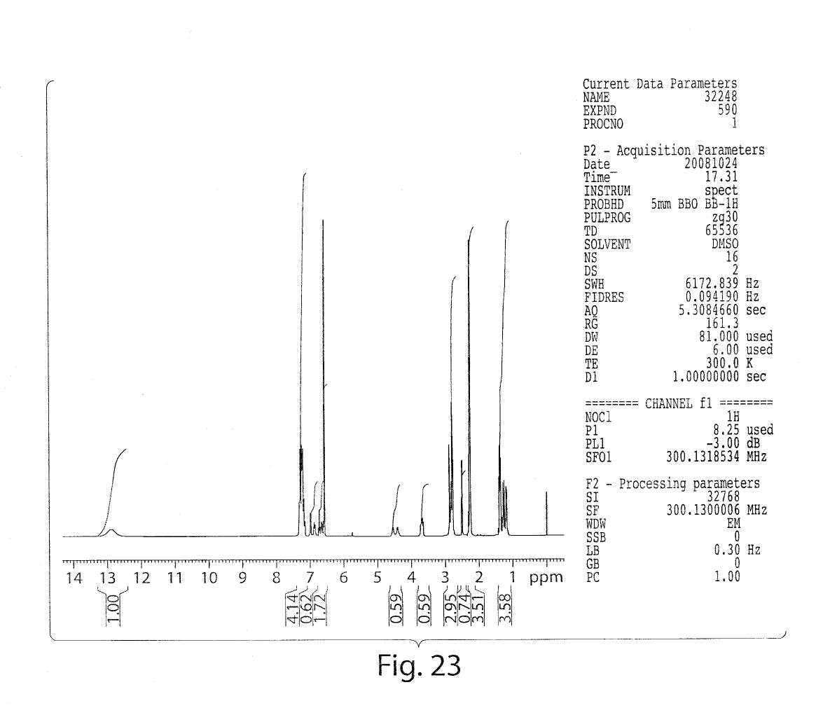

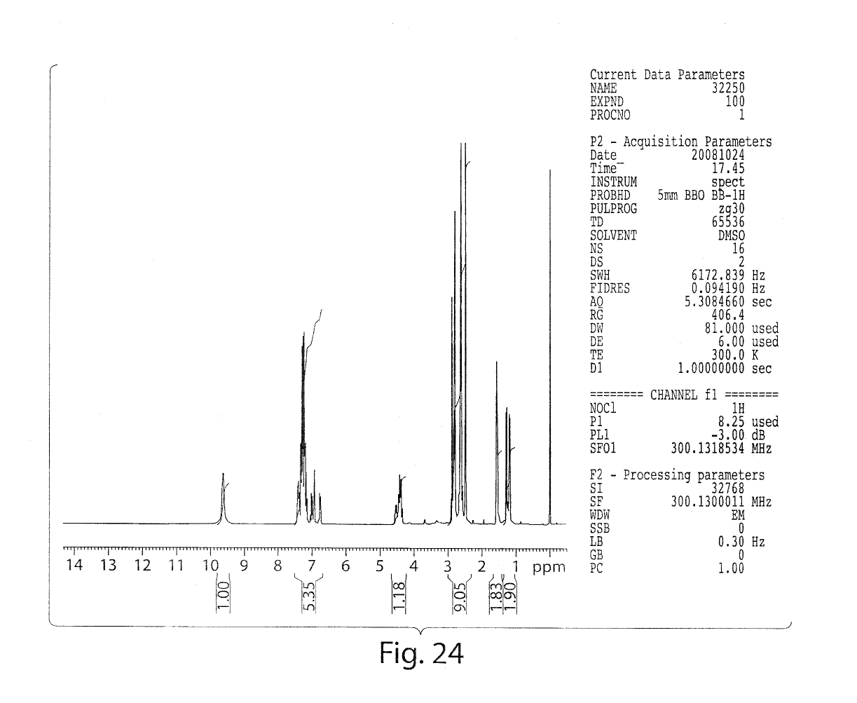

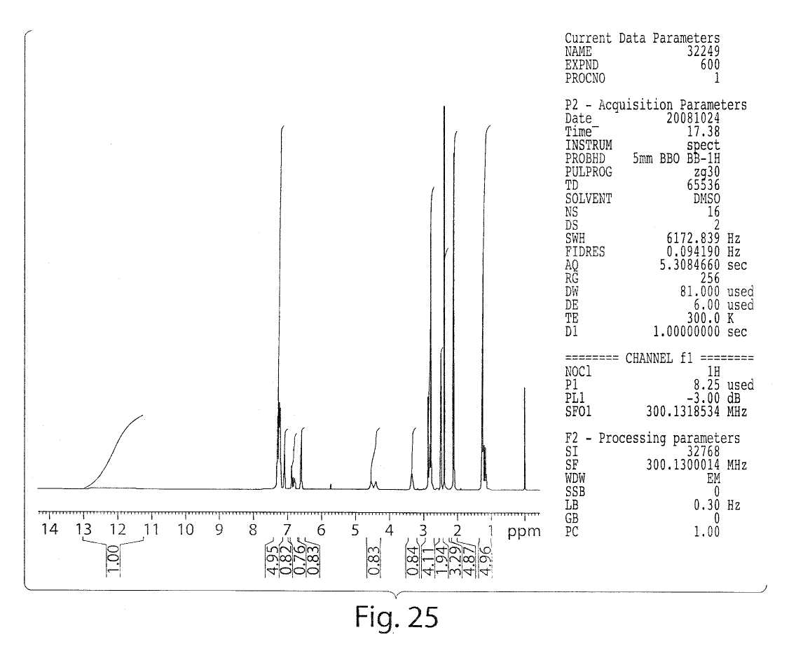

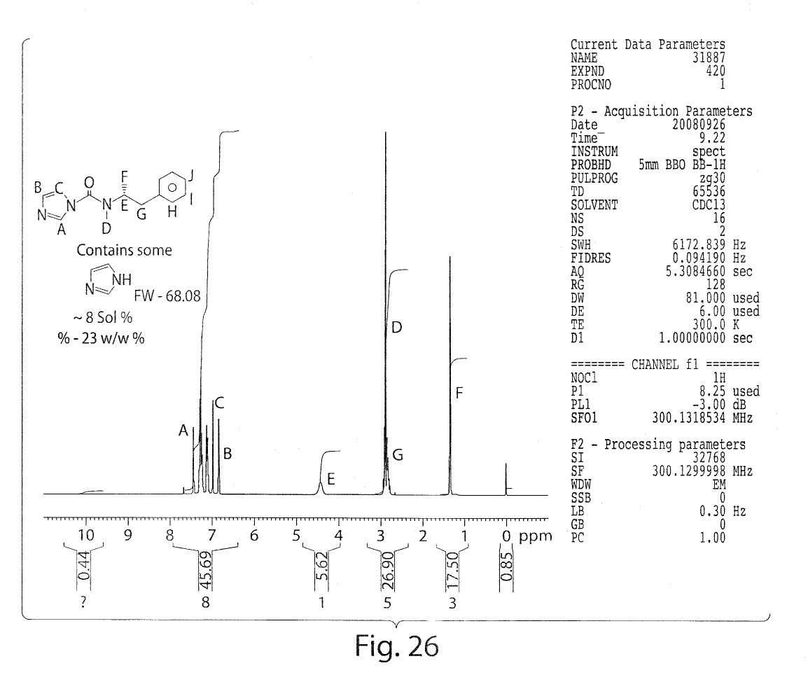

characterized by an X-ray diffraction pattern substantially similar to that set forth in FIG. 20. The present invention is directed to a hydrogen sulfate salt of compound 3d characterized by an x-ray diffraction pattern including characteristic peaks at about 10.1, 13.9, 14.9, 15.7, 18.1, 19.4, 19.9, 20.2, 21.3, 23.8, 24.2, and 26.1 degrees two-theta. The present invention is directed to a hydrogen sulfate salt of compound 3d:

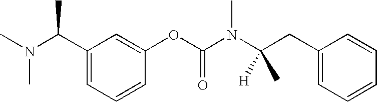

characterized by an x-ray diffraction pattern including characteristic peaks at about 10.1, 13.4, 13.9, 14.9, 15.7, 16.8, 17.1, 18.1, 19.4, 19.9, 20.2, 21.3, 22.2, 23.8, 24.2, 26.1, 26.6, and 27.4 degrees two-theta.

The present invention is directed to a hydrogen sulfate salt of compound 3d characterized by a Differential Scanning Calorimetry (DSC) thermogram having a single maximum value at about 112.02, as measured by a DSC Q100 V9.8 Build 96 instrument.

The present invention is directed to a hydrogen succinate salt of compound 3d:

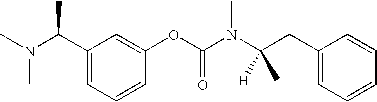

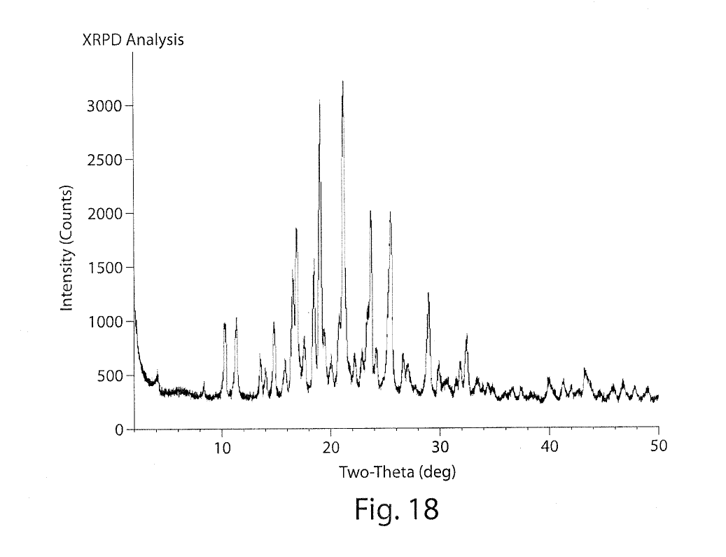

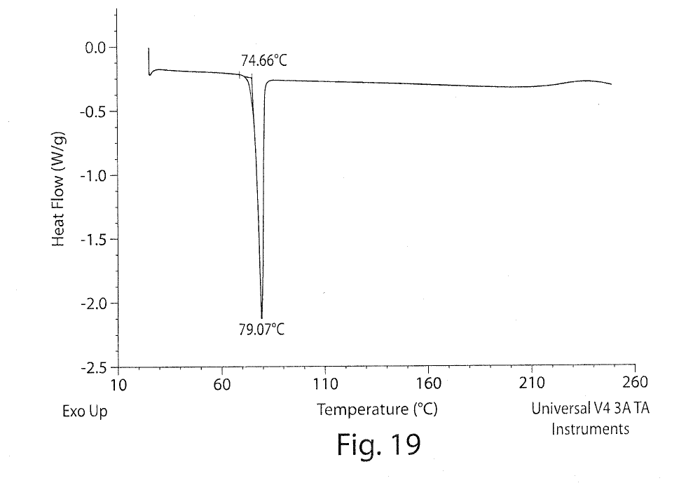

characterized by an x-ray diffraction pattern substantially similar to that set forth in FIG. 18. The present invention is directed to a hydrogen succinate salt of compound 3d characterized by an x-ray diffraction pattern including characteristic peaks at about 16.6. 16.9, 18.5, 19.1, 21.3, 23.4, 23.8, 25.6, and 29.0 degrees two-theta.

The present invention is directed to a hydrogen succinate salt of compound 3d characterized by an x-ray diffraction pattern including characteristic peaks at about 10.4, 11.4, 14.9, 16.6. 16.9, 18.5, 19.1, 21.3, 23.4, 23.8, 25.6, 29.0, and 32.5 degrees two-theta.

The present invention is directed to a hydrogen succinate salt of compound 3d:

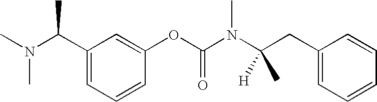

characterized by a Differential Scanning Calorimetry (DSC) thermogram having a single maximum value at about 79.07, as measured by a DSC Q100 V9.8 Build 96 instrument.

The compositions of the present invention comprise a compound or salt thereof that has cholinesterase activity. In one aspect, the compound or salt inhibits a cholinesterase by competing with a natural compound (e.g., acetylcholine (ACh) or butyrylcholine (BuCh)) that binds cholinesterase. The cholinesterase enzyme is inhibited when it is prevented from inactivating a natural compound, such as the neurotransmitter ACh, to any degree that cholinesterase would act on the neurotransmitter in the absence of the compound. The cholinesterase inhibited can be, for example, at least one member selected from the group consisting of an acetylcholinesterase (AChe) or a butyrylcholinesterase (BuChE). The compound or salt of the invention can inhibit AChE alone or BuChE alone or can inhibit both AChE and BuChE to similar or different degrees. AChE is located on excitable membranes and inactivates ACh. The excitable membrane can be a presynaptic neuron or a postsynaptic neuron. AChE is also referred to as specific cholinesterase. BuChE is located on excitable membranes and non-neuronal tissue such as blood cells. BuChE is also referred to as pseudocholinesterase or nonspecific cholinesterase. AChE and BuChE are regulators of cholinergic neurotransmission in the central nervous system (brain and spinal cord), peripheral nervous system and autonomic nervous system (parasympathetic nervous system and sympathetic nervous system).

A compound or salt of the invention can be useful as a CNS active agent, or for cardiovascular therapy, or as an antibacterial agent. For example, a CNS active agent is a useful for treating Alzheimer's disease, Parkinson's disease, attention deficit hyperactivity disorder (ADHD), depression, obsessive compulsive disorders (OCD), anxiety disorders, chronic pain, or narcolepsy. CNS active agents can be used to treat or prevent Alzheimer's disease, neuropathic pain, spasticity, Parkinson's disease,

Administration of a composition of the invention can, for example, result in an increase in ACh in the synapse of central nervous system neurons which can compensate for the cholinergic deficiency, for example, in Alzheimer's patients, thereby promoting neuronal transmission to ultimately alleviate or ameliorate the symptoms of Alzheimer's disease, Alzheimer's disease is accompanied by symptoms that include cognitive impairment, disoriented behavior, alter personality, difficulty speaking and comprehending and impaired gait and movement. It has been suggested that decreased cholinergic function is responsible for the symptoms of Alzheimer's disease (Benzi, G., et al., European J. Pharmacol. 346:1-13 (1998); Korczyn, A. D., Exp. Opin. Invest. Drugs 9:2259-2267 (2000)).

A compound or salt of the invention can decrease the amount of ACh synthesized or released, the inability of a neuron to respond to ACh or inactivation of AChE. In Alzheimer's disease, current treatments include the administration of compounds which increase cholinergic signaling (Jann, M. W., Pharmacotherapy 20:1-12 (2000); Bachurin, S. O., Med. Res. Rev. 23:48-88 (2003)). However, these compounds have modest efficacy, low response rate (typically about 30%-50%) and numerous side effects such as nausea, gastrointestinal problems and fatigue. In one embodiment, a compound or salt of the invention inhibits AChE and increases neurotransmitters, such as ACh, in the synapse of the central nervous system neurons. Thus, for example, the compound or salt of the invention inhibits AChE, which degrades ACh in the synapses of neurons in Alzheimer's patients, and increase neurotransmitters in the synapses.

Cholinergic deficiencies also characterize other disorders such as Parkinson's disease, progressive supranuclear palsy, vascular dementia and Down's syndrome (Korczyn, A. D., Exp. Opin. Invest. Drugs 9:2259-2267 (2000)). Thus, the composition of the invention can also be employed to increase the ACh in these disorders.

Likewise, administration of a composition of the invention can result in an increase in the neurotransmitter dopamine in the central nervous system of patients with Parkinson's disease, thereby promoting neuronal transmission to thereby diminish the symptoms of Parkinson's disease.

In one embodiment, the compound or salt of the invention is a memory-facilitating agent. In another embodiment, the compound or salt of the invention is a cognition-facilitating agent.

The term “memory-facilitating agent,” as used herein, refers to a compound or salt that promotes memory in an individual, prevents or minimizes a decline in memory in an individual or participates in biological processes which are involved in memory function.

The memory processes which can be facilitated by the memory-facilitating agent can be memory consolidation, the process of storing new information in long term memory (“Neuroscience: Exploring The Brain,” Bear, M. F. et al., Williams & Wilkins, Baltimore, Md., Ch. 19, pp. 517-545 (1996); McGaugh, J. L. Science 287: 248-251 (2000), the teachings of which are hereby incorporated by reference in their entirety); short-term memory (also referred to as “working memory”), the process whereby newly acquired information is maintained for short periods of time and the newly acquired information is made available for further information processing (“Neuroscience: Exploring The Brain,” Bear, M. F. et al., Williams & Wilkins, Baltimore, Md., Ch. 19, pp. 517-545 (1996); McGaugh, J. L. Science 287: 248-251 (2000); Becker, J. T., et al., Brain and Cognition 41:1-8 (1999), the teachings of which are hereby incorporated by reference in their entirety); declarative memory, which is the memory of facts and events (“Neuroscience: Exploring The Brain,” Bear, M. F. et al., Williams & Wilkins, Baltimore, Md., Ch. 19, pp. 517-545 (1996); McGaugh, J. L. Science 287: 248-251 (2000); Tulving, E., et al., Science 247: 301-306 (1990); Squire, L. R., et al., Proc. Natl. Acad. Sci. 93: 13515-13522 (1996), the teachings of which are hereby incorporated by reference in their entirety); procedural memory (also referred to as “tacit knowledge” or “implicit knowledge”), which is the memory for skills or behavior (“Neuroscience: Exploring The Brain,” Bear, M. F. et al., Williams & Wilkins, Baltimore, Md., Ch. 19, pp. 517-545 (1996); McGaugh, J. L. Science 287: 248-251 (2000), the teachings of which are hereby incorporated by reference in their entirety); or attention, acquisition, retrieval or retention.

In another embodiment, the compound or salt is a cognition-facilitating agent. The term “cognition-facilitating agent,” as used herein, refers to a compound or salt that promotes activities associated with thinking, learning and acquiring knowledge in an individual, prevents or minimizes a decline in thinking, learning and acquiring knowledge in an individual or participates in biological processes which are involved in thinking, learning and acquiring knowledge. The decline in thinking, learning and acquired knowledge (a cognitive disorder) can be a consequence of or associated with another disease (e.g., Alzheimer's disease) or condition of the central, or peripheral or autonomic nervous system. The cognitive process that can be facilitated by the cognition-facilitating agent can be assessed by behavioral criteria and behavioral assays which, in turn, can further define where, in the learning, thinking, and acquiring knowledge process, the cognition-facilitating agents are acting.

An “agent,” as used herein, refers to a compound that can produce a physical, chemical or biological effect that can be stimulatory (e.g., an activating agent) or inhibitory (e.g., a blocking agent). Agents that are stimulatory can be agonists. Agents that are inhibitory can be antagonists or inverse agonists. Inverse agonists are compounds that down-regulate receptor activated activity thereby acting in a manner that is the opposite of an agonist to the receptor. Thus, exposure or administration of an inverse agonist can result in a diminished response compared to exposure or administration of an agonist.

A cholinergic agent can be, for example, a compound that stimulates the action of ACh thereby mediating ACh-mediated cell signaling between two cells (a cholinergic agonist). Stimulation can be, for example, a result of facilitating binding of ACh to a cell surface receptor, interference with degradation of ACh, stimulation of release of ACh, stimulation of synthesis of ACh, activation of second messengers (e.g., phospholipase C, inositol 1,4,5-triphosphate, protein kinase C, protein kinase A) that mediate ACh cell signaling, alteration of ion (e.g., sodium, potassium) channels in target cells. An agent can also inhibit or prevent any one or more of these effects (e.g., a cholinergic antagonist).

The compound or salt of the invention can inhibit cholinesterase activity, which can be expressed as an IC50. The term “IC50,” as used herein, refers to the concentration of a compound that inhibits an activity or effect by 50%, e.g., by reducing the frequency of a condition, such as memory or cognitive loss by 50%; by reducing binding of a competitor molecule to a protein (e.g., a receptor) by 50%; or by reducing the level of an activity (e.g., cholinesterase activity) by 50%.

As used herein, an “individual” is any mammal. A mammal can be a rodent (such as a rat, mouse or guinea pig), domesticated animal (such as a dog or cat), ruminant animal (such as a horse or a cow) or a primate (such as a monkey or a human). In a preferred embodiment, the individual is a human.

An individual suffering from a condition can be treated by a composition of the invention. For example, the condition can include at least one condition selected from the group consisting of a central nervous system condition, a peripheral nervous system condition and an autonomic nervous system condition.

In a particular embodiment, the individual treated with a composition of the invention has a central nervous system condition. A “central nervous system condition,” as used herein, refers to any illness or ailment that affects the brain or spinal cord of the individual. Central nervous system conditions treated with a composition of the invention, can, for example, be a consequence of a genetic disease, environmental exposure to a compound or secondary to a primary illness or disease. The central nervous system condition can be characterized by or a consequence of inadequate neurotransmitter release, synthesis, processing, re-uptake or cell signaling. The central nervous system condition can additionally, or alternatively, be characterized by or a consequence of failed or inadequate neuronal transmission due to disruptions in ion channels.

In a particular embodiment, a central nervous system condition is treated with a composition of the invention. The composition of the invention can be used to treat conditions such as depression, anxiety or mental conditions. Compounds of the invention can be used to treat conditions such as Parkinson's disease, a memory impairment or a cognitive impairment.

The memory impairments can be in a human individual. Memory impairments that can be treated by the compounds of the invention include Alzheimer's disease, age-associated memory loss, an impairment in memory consolidation, an impairment in short term memory, mild cognitive impairment, an impairment in declarative memory and/or impairments in memory associated with or a consequence of multiple sclerosis and/or Parkinson's disease.

The memory impairment treated by the compositions of the invention can be a consequence of exposure to a muscarinic cholinergic receptor antagonist. In one embodiment, the muscarinic cholinergic receptor antagonist is atropine. In another embodiment, the muscarinic cholinergic receptor antagonist is scopolamine. In yet another embodiment, the muscarinic cholinergic receptor antagonist is homatropine.

A muscarinic cholinergic receptor antagonist includes any substance which blocks, diminishes, attenuates, inhibits, hinders, limits, decreases, reduces, restricts or interferes with the action of ACh thereby disrupting ACh-mediated cell signaling between presynaptic and postsynaptic neurons. The antagonist can, for example, oppose the action of ACh by acting in a manner which prevents ACh from binding to a muscarinic cholinergic receptor on a postsynaptic neuron, from mediating post-synaptic events following binding of ACh to a muscarinic cholinergic receptor, interfere with ACh degradation by acetylcholinesterase in the synaptic cleft or interfere with release of ACh from presynaptic neurons.

In still another embodiment, the compositions of the invention can be used to treat a peripheral nervous system condition in an individual. The peripheral nervous system condition can, for example, be a disease or illness consequent to or associated with neurons which supply innervation to a skeletal muscle (e.g., Myasthenia Gravis). Conditions of the peripheral nervous system can be, for example, an impairment in the release of acetylcholine from neurons at the neuromuscular junction of skeletal, smooth or cardiac muscle.

The compositions of the invention can be used to treat an autonomic nervous system condition (sympathetic nervous system, parasympathetic nervous system) in an individual. The autonomic nervous system conditions can be conditions which affect smooth muscle of viscera, glands (endocrine glands, exocrine glands), blood vessels or cardiac muscle. Autonomic nervous system conditions treated employing the compounds of the invention can be post-operative distension and urinary retention. Conditions of the autonomic nervous system can be an impairment in a function associated with the autonomic nervous system, for example, an impairment in the release of norepinephrine from sympathetic neurons or ACh from parasympathetic neurons at a synapse with a cell (e.g., epithelial, nervous, muscle, connective tissue) in an organ, blood vessel or gland. One skilled in the art would be capable of diagnosing an individual with a central nervous system condition, peripheral nervous system condition and an autonomic nervous system condition.

An “impairment in memory or cognition,” as used herein, refers to a diminished capacity in memory and/or cognitive processes in the human. The cognitive and/or memory processes and impairments in cognitive and/or memory processes can be assessed or determined by established techniques. For example, memory can be assessed before, concomitantly with or after treatment of the individual with a composition of the invention one or more well established tests known to one of skill in the art. Such tests include the Passive Avoidance Testing (Principles of Neuropsychopharmacology), Feldman R. S., et al., Sinauer Assoc., Inc., Sunderland, Mass. (1997), the teachings of all of which are incorporated by reference in their entirety); Rey Auditory Verbal Learning Test (RAVLT) (L′ examen clinique en psychologie), Rey A., Paris: Presses Universitaires de France (1964); a Wechsler Memory Scale; Wechsler Memory Scale-Revised (Wechsler, D., Wechsler Memory Scale-Revised Manual, NY, N.Y., The Psychological Corp. (1987)); California Verbal Learning Test-Second Edition (Delis, D. C., et al., The Californian Verbal Learning Test, Second Edition, Adult Version, Manual, San Antonio, Tex.: The Psychological Corporation (2000)); Cognitive Drug Research (CDR) Computerized Assessment Battery-Wesnes; Buschke's Selective Reminder Test (Buschke, H., et al., Neurology 24: 1019-1025 (1974)); Brief Visuospatial Memory Test-Revised; and Test of Everyday Attention (Perry, R. J., et al., Neuropsychologia 38: 252-271 (2000)).

In a particular embodiment, the memory of the human before, during or after administration of the composition of the invention is assessed or determined by a word recall test such as RAVLT.

In another embodiment, the invention described herein provides a method of treating a nervous system condition in an individual. The method includes administering to the individual a composition of the invention. The compound of the composition inhibits a cholinesterase thereby treating the nervous system condition of the individual.

In a particular embodiment, administration of the compound of the invention treats a central nervous system condition in an individual. In one aspect, the compound inhibits acetylcholinesterase thereby treating the central nervous system condition in the individual. The compound, upon hydrolysis, e.g., by reaction with the acetylcholinesterase, becomes at least one component of a pharmacologically active agent that further treats the central nervous system condition in the individual.

A further embodiment of the invention is a method of increasing acetylcholine in an in vitro sample. The method includes administering to the in vitro sample a compound. The compound inhibits a cholinesterase, thereby increasing acetylcholine in the in vitro sample.

The in vitro sample can be a cell-free sample or a sample containing cells. The cells employed can be mammalian cells (e.g., CHO cells), insect cells or bacterial cells. The method can be employed to assess the ability of the compound to inhibit cholinesterase and the pharmacologically active agent to affect biological, chemical or physical processes prior to use in an individual. The method can be packaged in a kit as an assay for screening the compounds of the invention for cholinesterase activity and pharmacological activity of the agents the compound becomes upon hydrolysis.

Another embodiment of the invention is a method of increasing acetylcholine in a tissue. The method includes administering to the tissue a composition of the invention. The compound of the composition inhibits a cholinesterase, thereby increasing acetylcholine in the tissue and, upon hydrolysis, e.g., by reaction with the cholinesterase, becomes at least a component of a pharmacologically active agent that further increases acetylcholine in the tissue.

The tissue can be a nervous tissue, a muscle tissue (cardiac, skeletal, smooth muscle) or a collection of any one or more of a tissue type selected from the group consisting of nervous tissue, muscle tissue, epithelial tissue and connective tissue. The tissue can be isolated (removed from the individual).

An additional embodiment of the invention is a method of increasing acetylcholine in an individual. The method includes administering to the individual a composition of the invention in the individual. The composition inhibits a cholinesterase (e.g., AChE, BuChE), thereby increasing acetylcholine.

In one embodiment, the compound or salt of the invention increases acetylcholine in the central nervous system of the individual. In another embodiment, the compound or salt of the invention increases acetylcholine in the peripheral nervous system of the individual. In yet another embodiment, administration of a composition of the invention increases acetylcholine in the autonomic nervous system of the individual. Techniques to assess the increase of ACh in an in vitro sample, in a tissue and in an individual are well-known to one skilled in the art. (See, for example, Day, J. C., et al. Methods 23:21-39 (2001), the teachings of which are hereby incorporated by reference in its entirety).

The further increase in acetylcholine can be an increase mediated in a manner similar to the increase mediated by the compound of the invention (inhibition of AChE) or an increase in ACh by, for example, increasing the release of ACh, increasing the synthesis of ACh or otherwise preventing the inactivation of ACh.

In a further embodiment, the invention is a method of increasing transmission between two or more neurons. The method includes exposing the neurons to a compound of the invention. The compound inhibits a cholinesterase, thereby increasing transmission between the two or more neurons.

The transmission can be increased between two or more neurons in vitro or in vivo. Techniques to determine an increase in transmission in vitro and in vivo are well known to one skilled in the art. For example, changes in depolarization of the post-synaptic neuron can be recorded by electrophysiological methods.

An increase in transmission in an individual can minimize or alleviate central or peripheral nervous system conditions, such as memory and cognitive impairments. For example, an increase in cholinergic transmission (e.g., post-synaptic) in a human individual can minimize or alleviate the symptoms associated with Alzheimer's disease. An increase in dopaminergic transmission (e.g., post-synaptic) in a human individual can minimize or alleviate the symptoms associated with Parkinson's disease. The compound of the invention can facilitate penetration of the compound through the blood brain barrier, thereby permitting delivery of a pharmacologically active agent, in particular, into the central nervous system.

Another embodiment of the invention is a method of treating a cholinergic deficiency in an individual. The method includes administering to the individual a composition of the invention. The composition of the invention inhibits a cholinesterase thereby treating the cholinergic deficiency in the individual.

The cholinergic deficiency can be a nervous system deficiency. For example, the composition of the invention can be used to treat a human individual having Alzheimer's disease. Presynaptic neurons degenerate rapidly in Alzheimer's disease which limits the efficacy of ChE inhibition as the disease progresses (Cutler, N. R., et. al. CNS Drugs 3:467-481 (1995)). ChE continues to be present in the synapses of neurons in an individual with Alzheimer's disease, hydrolyzing what little ACh may be present in the synapse. Thus, the compounds of the invention can become a cholinergic agonist thereby ameliorating the cholinergic deficiency by increasing ACh-mediated synaptic transmission in the central nervous system of individuals suffering from Alzheimer's disease, mild cognitive impairment, age associated memory impairment, age associated memory loss, natural aging, vascular dementia, dementia with Lewis bodies and/or Parkinson's disease.

In an additional embodiment, the invention is a method of treating an impairment in memory in an individual. The method includes administering to the individual a composition of the invention. The compound inhibits a cholinesterase thereby treating the impairment in memory in the individual.

The memory impairment can be a memory impairment selected from the group consisting of an impairment in memory consolidation, an impairment in long-term memory and an impairment in short-term memory. One skilled in the art would be capable of identifying an individual with a memory impairment and assessing the impairment.

In a particular embodiment, a human individual has an impairment in memory associated with a condition selected from the group consisting of Alzheimer's disease, Parkinson's disease, age-associated memory loss, mild cognitive impairment and multiple sclerosis.

In another embodiment the human individual treated with the compound of the invention has age-related cognitive decline.

A further embodiment of the invention is a method of delivering a pharmacologically active agent to a tissue. The term “pharmacologically active agent,” as used herein, refers to a compound that influences biological processes by altering the activity, localization and/or expression of molecules (e.g., neurotransmitters, peptides, proteins) which are directly or indirectly involved in the biological processes. The method includes administering to the tissue a compound of the invention. The compound of the invention inhibits a cholinesterase and, upon hydrolysis, e.g., by reaction with the cholinesterase, becomes at least a component of a pharmacologically active agent, thereby delivering the pharmacologically active agent to the tissue.

The tissue can be an in vitro tissue sample or can be a tissue in vivo (in an individual). The tissue can be muscle tissue, nervous tissue or any combination of muscle, nervous, connective or epithelial tissue. The compound of the invention can be employed to deliver a pharmacologically active agent to a tissue that is proximal or distal to a tissue having a cholinesterase that is inhibited by the compound of the invention. For example, a compound of the invention can be employed to deliver a pharmacologically active agent, such as a cholinergic agent, to a muscle tissue. The compound of the invention can bind a cholinesterase (acetylcholinesterase, butyrylcholinesterase) thereby inhibiting the activity of the cholinesterase and, upon hydrolysis (with, for example, a cholinesterase), become a cholinergic agent. The pharmacologically active agent can be delivered to a muscle cell proximate to the site of binding of the compound of the invention to the cholinesterase or to a muscle cell distal to the site of binding. Similarly, the compound can bind to a cholinesterase in a neuron of the nervous system and deliver a cholinergic agent proximal or distal to the site of binding.

The compound of the invention can bind to a cholinesterase and, upon hydrolysis, e.g., by reaction with the cholinesterase, deliver methamphetamine to a neuron proximate or distal to the site of binding of the compound of the invention. Thus, the compounds of the invention provide a method of delivering a pharmacologically active agent to the central nervous system. The pharmacologically active agents can diffuse to varying regions of the brain and mediate their effects.

The invention includes a method of treating a condition of an individual by administering a composition of the invention, wherein the condition is a nervous system condition selected from delayed gastric emptying, attention deficit hyperactivity disorder (ADHD), phobia, sleep disorder, stroke, psychiatric disorder, pain, anticholinergic drug overdose, tobacco dependence, Parkinson's disease, memory impairment, and cognitive impairment. The invention includes use of a composition of the invention in the manufacture of a medicament for treating a condition of an individual, wherein the condition is a nervous system condition selected from delayed gastric emptying, attention deficit hyperactivity disorder (ADHD), phobia, sleep disorder, stroke, psychiatric disorder, pain, anticholinergic drug overdose, tobacco dependence, Parkinson's disease, memory impairment, and cognitive impairment.

The invention includes a method of treating a condition in an individual by administering a composition of the invention, wherein the condition is selected from glaucoma, oncologic condition, delayed gastric emptying, attention deficit hyperactivity disorder (ADHD), phobia, stroke, multiple sclerosis, sleep disorder, psychiatric disorder, pain, anticholinergic drug overdose, tobacco dependence, and spasticity. The invention includes use of a composition of the invention in the manufacture of a medicament for treating a condition in an individual, wherein the condition is selected from glaucoma, delayed gastric emptying, attention deficit hyperactivity disorder (ADHD), phobia, stroke, multiple sclerosis, sleep disorder, psychiatric disorder, pain, anticholinergic drug overdose, tobacco dependence, and spasticity.

In one embodiment, the invention includes a method of treating a condition of an individual by administering a composition of the invention, wherein the condition is selected from delayed gastric emptying, attention deficit hyperactivity disorder (ADHD), phobia, sleep disorder, stroke, psychiatric disorder, pain, anticholinergic drug overdose, and tobacco dependence. The invention includes use of a composition of the invention in the manufacture of a medicament for treating a condition of an individual, wherein the condition is selected from delayed gastric emptying, attention deficit hyperactivity disorder (ADHD), phobia, sleep disorder, stroke, psychiatric disorder, pain, anticholinergic drug overdose, and tobacco dependence.

In one embodiment, the invention includes a method of treating a condition of an individual by administering a composition of the invention, wherein the condition is anticholinergic drug overdose. The invention includes use of a compound of the invention in the manufacture of a medicament for treating a condition of an individual, wherein the condition is anticholinergic drug overdose.

In one embodiment, the invention includes the methods discussed above, wherein the individual is a human.

Another aspect of the invention includes a method of promoting wakefulness in an individual by administering a composition of the invention, wherein the individual suffers from a disorder or condition selected from wakefulness disorders, hypersomnia, sleep apnea, sleep disorders of central origin, fatigue, excessive daytime sleepiness associated with narcolepsy, fatigue and excessive sleepiness associated with a depressive disorder or with antidepressant therapy.

Another aspect of the invention includes a method of promoting wakefulness, thereby treating the individual for a disorder or condition selected from a wakefulness disorder, hypersomnia, sleep apnea, sleep disorder of central origin, fatigue, excessive daytime sleepiness associated with narcolepsy, fatigue and excessive sleepiness associated with a major depressive disorder or with antidepressant therapy.

In one aspect, the invention includes a method for the treatment of a wakefulness disorder by administering to composition of the invention as a wake promoting agent. In one aspect, the invention includes a method for the treatment of sleep apnea by administering to an individual a composition of the invention as a wake promoting agent. In one aspect, the invention includes a method for the treatment of a sleep disorder of central origin by administering to an individual a composition of the invention as a wake promoting agent. In one aspect, the invention includes a method for the treatment of fatigue by administering to an individual a composition of the invention as a wake promoting agent. In one aspect, the invention includes a method for the treatment of excessive daytime sleepiness associated with narcolepsy by administering to an individual a composition of the invention as a wake promoting agent. In one aspect, the invention includes a method for the treatment fatigue and excessive sleepiness associated with a major depressive disorder by administering to an individual a composition of the invention as a wake promoting agent. In one aspect, the invention includes a method for the treatment fatigue and excessive sleepiness associated with antidepressant therapy.

Fatigue and excessive sleepiness are among the symptoms of a major depressive disorder, and can be adverse experiences associated with antidepressant therapy and are often residual symptoms inadequately treated with SSRI antidepressant therapy. Antidepressant therapy includes but is not limited to therapy with the following antidepressants: tricyclic antidepressants, selective serotonin reuptake inhibitors (SSRIs), serotonin and noradrenaline reuptake inhibitors, monoamine oxidase inhibitors and monoamine oxidase type A. In another aspect, antidepressant is selected from citalopram, fluoxetine, fluoxetine hydrochloride, paroxetine, paroxetine hydrochloride, and clomipramine hydrochloride.

In one aspect, the invention relates to hypersomnia, a condition that is characterized by reoccurring episodes of excessive daytime sleepiness (EDS) or prolonged nighttime sleep. Different from feeling tired due to lack of or interrupted sleep at night, persons with hypersomnia are compelled to nap repeatedly during the day, often at inappropriate times such as at work, during a meal, or in conversation. These daytime naps usually provide no relief from symptoms. Patients often have difficulty waking from a long sleep, and may feel disoriented. Other symptoms may include anxiety, increased irritation, decreased energy, restlessness, slow thinking, slow speech, loss of appetite, hallucinations, and memory difficulty. Some patients lose the ability to function in family, social, occupational, or other settings. In one aspect, the invention includes a method for the treatment of hypersomnia, which comprises administering to an individual a composition of the invention as a wake promoting agent. In another aspect, the invention includes a method for the treatment of hypersomnia, which comprises administering to an individual a composition of the invention as an arousing agent.

In another aspect, the invention includes a method of promoting wakefulness, wherein the wakefulness disorder or condition is selected from circadian rhythm disorder and fatigue associated with multiple sclerosis.

In one aspect, the invention includes a method of promoting wakefulness by administering a composition of the invention, wherein the circadian rhythm disorder is selected from shift work sleep disorder, sleep apnea, desynchronizing disorder in blind individuals, time zone change syndrome, shift work sleep disorder, irregular sleep pattern, delayed sleep syndrome, and advanced sleep syndrome. In another aspect, the invention includes a method of promoting wakefulness, wherein the circadian rhythm disorder is selected from shift work sleep disorder, sleep apnea, and desynchronizing disorder in blind individuals.

In one aspect, the invention relates to sleep apnea. Sleep apnea is a sleep disorder characterized by pauses in breathing during sleep. Each episode, called an apnea, lasts long enough so that one or more breaths are missed, and such episodes occur repeatedly throughout sleep. The standard definition of any apneic event includes a minimum 10 second interval between breaths, with either a neurological arousal (a 3-second or greater shift in EEG frequency, measured at C3, C4, O1, or O₂), a blood oxygen desaturation of 3-4% or greater, or both arousal and desaturation. Sleep apnea is diagnosed with an overnight sleep test called a polysomnogram.

Clinically significant levels of sleep apnea are defined as five or more episodes per hour of any type of apnea (from the polysomnogram). There are three distinct forms of sleep apnea: central, obstructive, and complex (i.e., a combination of central and obstructive) constituting 0.4%, 84% and 15% of cases respectively. Breathing is interrupted by the lack of respiratory effort in central sleep apnea; in obstructive sleep apnea, breathing is interrupted by a physical block to airflow despite respiratory effort. In complex (or “mixed”) sleep apnea, there is a transition from central to obstructive features during the events themselves.

In one aspect, the invention includes a method for the treatment of sleep disorders of central origin by administering to an individual a composition of the invention. In another aspect, the invention includes a method for the treatment of sleep disorders of central origin by administering to an individual a composition of the invention, wherein the number of apneas occurring during sleep apnea syndromes is reduced. In one aspect, treatment of sleep disorders of central origin by administering a composition of the invention contributes to improving diurnal somnolence and the quality of nocturnal sleep.

In one aspect, the invention includes a method of promoting wakefulness in an individual, wherein individual is being treated for sleep apnea with CPAP. “CPAP” or “continuous positive airway pressure” is a mechanical device for the treatment for sleep apnea and other sleep-related breathing disorders (including snoring). Treatment with a CPAP device is typically administered via the nose or mouth of the patient.

Under CPAP treatment, a subject wears a tight-fitting plastic mask over the nose when sleeping. The mask is attached to a compressor, which forces air into the nose creating a positive pressure within the subject's airways. The principle of the method is that pressurizing the airways provides a mechanical “splinting” action, which prevents or lessens airway collapse and therefore, obstructive sleep apnea. Although an effective therapeutic response is observed in most subjects who undergo CPAP treatment, many subjects cannot tolerate the apparatus or pressure and refuse treatment. Moreover, recent covert monitoring studies demonstrated that long-term compliance with CPAP treatment is very poor. It is known that subjects remove their mask while sleeping.

In another aspect, the invention relates to fatigue associated with multiple sclerosis (MS). Multiple sclerosis is one of the most common disabling neurologic diseases of young adults in the United States, where an estimated 400,000 persons have the disease. Although MS can cause a variety of disabling neurological impairments such as blindness, paralysis, incoordination, and bowel or bladder dysfunction, a less apparent symptom that can also be severely disabling is fatigue. As used herein “fatigue” includes loss of power, or capacity to respond to stimulation. Effect treatment of such fatigue includes alleviating tiredness, or sleepiness associated with multiple sclerosis and also promoting wakefulness in multiple sclerosis individuals. The mechanism of MS fatigue is poorly understood. It has been attributed to nerve conduction abnormalities within the central nervous system and increased energy demands caused by neurologic disability. Several characteristics of MS fatigue are interference with physical functioning and activities of daily living, aggravation by heat, and worsening at the end of the day. In aspect, the invention includes a method of treatment for fatigue associate with multiple sclerosis, comprising administering to an individual a composition of the invention to improve or prevent symptoms of multiple sclerosis fatigue in the individual. In another aspect, the invention includes alleviating tiredness, or sleepiness associated with multiple sclerosis and also promoting wakefulness in multiple sclerosis individuals.

One aspect of the invention includes a method for enhancing alertness or increasing regularity of sleep rhythms in an individual by administering a composition of the invention.

In one aspect, the invention includes a method of promoting wakefulness, wherein the compound or salt of the composition administered has a reduced abuse potential. In one aspect of the invention, no psychostimulant-like effects are observed in the individual following administration of the composition of the invention. A psychostimulant is a drug that causes a sense of well-being, decreases fatigue and depression, and increases the desire to eat. Psychostimulant drugs can also cause mood changes and trouble with sleeping. In another aspect of the invention, the composition of the invention administered has a dose-limiting side effect. In one aspect, of the invention, the side effect is nausea.

In another aspect of the invention, administration of the composition of the invention does not cause rebound hypersomnolence in the individual. The term “hypersomnolence” refers to an excessive need for sleep, especially during the day. “Idiopathic hypersomnolence” means a need for excessive daytime sleep without a known cause. In another aspect of the invention, administration of the composition of the invention does not cause psychostimulant effects.

Another aspect of the invention includes the manufacture of a medicament for promoting wakefulness in an individual that suffers from a disorder or condition selected from wakefulness disorders, hypersomnia, sleep apnea, sleep disorders of central origin, fatigue, excessive daytime sleepiness associated with narcolepsy, fatigue and excessive sleepiness associated with a depressive disorder or with antidepressant therapy, wherein said medicament comprises a composition described herein.

Another aspect of the invention includes the manufacture of a medicament for promoting wakefulness in an individual and thereby treating the individual a disorder or condition selected from wakefulness disorders, hypersomnia, sleep apnea, sleep disorders of central origin, fatigue, excessive daytime sleepiness associated with narcolepsy, fatigue and excessive sleepiness associated with a depressive disorder or with antidepressant therapy, wherein said medicament comprises a composition described herein.

The present invention is directed to a process for preparing compound 3d: 3-((S)-1-(dimethylamino)ethyl)phenyl methyl-((R)-1-phenylpropan-2 yl)carbamate comprising the steps of:

(1) reacting L-methamphetamine with carbonyldiimidazole to form (R)—N-Methyl-N-(1-phenylpropan-2-yl)-1H-imidazole-1-carboxamide; and

(2) reacting (R)—N-Methyl-N-(1-phenylpropan-2-yl)-1H-imidazole-1-carboxamide with (S)-rivalphenol to yield 3-((S)-1-(dimethylamino)ethyl)phenyl methyl-((R)-1-phenylpropan-2 yl)carbamate.

In one embodiment, the invention is a process for preparing the hydrogen fumarate of compound 3d: 3-((S)-1-(dimethylamino)ethyl)phenyl methyl-((R)-1-phenylpropan-2 yl)carbamate hydrogen fumarate comprising the steps of:

(1) reacting L-methamphetamine with carbonyldiimidazole to form (R)—N-Methyl-N-(1-phenylpropan-2-yl)-1H-imidazole-1-carboxamide;

(2) reacting (R)—N-Methyl-N-(1-phenylpropan-2-yl)-1H-imidazole-1-carboxamide with (S)-rivalphenol to yield 3-((S)-1-(dimethylamino)ethyl)phenyl methyl-((R)-1-phenylpropan-2 yl)carbamate; and

(3) contacting 3-((S)-1-(dimethylamino)ethyl)phenyl methyl-((R)-1-phenylpropan-2 yl)carbamate with fumaric acid to yield 3-((S)-1-(dimethylamino)ethyl)phenyl methyl-((R)-1-phenylpropan-2 yl)carbamate hydrogen fumarate.

In another embodiment, the invention is a process for preparing 3-((S)-1-(dimethylamino)ethyl)phenyl methyl-((R)-1-phenylpropan-2 yl)carbamate hydrogen succinate comprising the steps of:

(1) reacting L-methamphetamine with carbonyldiimidazole to form (R)—N-Methyl-N-(1-phenylpropan-2-yl)-1H-imidazole-1-carboxamide;

(2) reacting (R)—N-Methyl-N-(1-phenylpropan-2-yl)-1H-imidazole-1-carboxamide with (S)-rivalphenol to form 3-((S)-1-(dimethylamino)ethyl)phenyl methyl-((R)-1-phenylpropan-2 yl)carbamate; and

(3) contacting 3-((S)-1-(dimethylamino)ethyl)phenyl methyl-((R)-1-phenylpropan-2 yl)carbamate with succinic acid to yield 3-((S)-1-(dimethylamino)ethyl)phenyl methyl-((R)-1-phenylpropan-2 yl)carbamate hydrogen succinate.

In yet another embodiment, the invention is a process for preparing 3-((S)-1-(dimethylamino)ethyl)phenyl methyl-((R)-1-phenylpropan-2 yl)carbamate hydrogen sulfate comprising the steps of:

(1) reacting L-methamphetamine with carbonyldiimidazole to form (R)—N-Methyl-N-(1-phenylpropan-2-yl)-1H-imidazole-1-carboxamide;

(2) reacting (R)—N-Methyl-N-(1-phenylpropan-2-yl)-1H-imidazole-1-carboxamide with (S)-rivalphenol to form 3-((S)-1-(dimethylamino)ethyl)phenyl methyl-((R)-1-phenylpropan-2 yl)carbamate; and

(3) contacting 3-((S)-1-(dimethylamino)ethyl)phenyl methyl-((R)-1-phenylpropan-2 yl)carbamate with sulfuric acid to yield 3-((S)-1-(dimethylamino)ethyl)phenyl methyl-((R)-1-phenylpropan-2 yl)carbamate hydrogen sulfate.

In one aspect, the process comprising the step of recrystallizing a crude preparation of the salt (e.g., fumarate, sulfate, succinate) of compound 3d from an organic solvent or a mixture of an organic solvent and water. In a particular embodiment, the salt of compound 3d is produced by a process comprising the step of recrystallizing a crude preparation of the compound 3d from an organic solvent. The organic solvent may be an acetate such as isopropyl acetate. In another embodiment, the salt of compound 3d is produced by a process comprising the step of recrystallizing a crude preparation of the compound 3d from a mixture of isopropyl acetate (IPA) and acetone.

Compounds of the invention relate to rivastigmine-based conjugates of amphetamine isomers. Rivastigmine was chosen as starting point for the compounds because it has proven clinical utility in the management of Alzheimer's disease and because the corresponding phenol is chemically well characterized, accessible and stable. Amphetamines were selected due to their attractive and well characterized pharmacological properties. Amphetamines are neurotransmitter uptake inhibitors (Creese, I. and S. D. Iversen, The pharmacological and anatomical substrates of the amphetamine response in the rat. Brain Res., 1975. 83: p. 419-436) with stimulant properties (Wise, R. A. and P. P. Rompre, Brain dopamine and reward Ann. Rev. Psychol., 1989. 40: p. 191-225; Mason, S. T., The neurochemistry and pharmacology of extinction behavior. Neurosci. Biobehav. Rev., 1983. 7: p. 325-347) which could be beneficial in the treatment of (geriatric) depression and fatigue, two symptoms that often accompany Alzheimer's and Parkinson's disease. In addition, dextro-amphetamine is a well known cognitive

The bifunctional cholinesterase inhibitors of the invention can be prepared through combination of a pharmacologically active amine and the phenol moiety of a known cholinesterase inhibitor in a single molecule. The resulting hybrid molecules retain their ability to inhibit cholinesterase, both in vitro and in vivo, and as demonstrated, release pharmacologically active amines following decarbamylation of the inhibited enzyme.

The high level of brain and plasma cholinesterase inhibition in absence of severe side effects following oral doses of a compound or salt of the invention is unprecedented and suggests that the bifunctional cholinesterase inhibitors of the invention may have a greater therapeutic window than currently known cholinesterase inhibitors. Since it can be expected that peripheral cholinergic effects are antagonized by the release of adrenergic agents, the increased tolerability may be a reflection of in vivo pharmacological effects of released d-amphetamine. One aspect of the present invention is directed to a compound having the structure

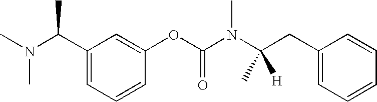

or a pharmaceutically acceptable salt thereof.

### DEFINITIONS

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, and toluene sulfonic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., USA, p. 1445 (1990).

The term “wake promotion” or “promoting wakefulness” as used herein, refers to a marked increase in the duration of wakefulness of an individual. In one aspect, there is no rebound hypersomnolence in an individual to whom a composition of the invention is administered. In one aspect, there is a reduction in drowsiness i.e., there is an increased state of mental alertness, or the prevention of further progression into a deeper state of drowsiness that prefaced administration of a composition of the invention. The term “drowsiness” is art-recognized, including decreased states of mental alertness.

As used herein, “treating” or “treatment” includes any effect e.g., lessening, reducing, modulating, or eliminating, that results in the improvement of the condition, disease, disorder, etc. “Treating” or “treatment” of a disease state means the treatment of a disease-state in a mammal, particularly in a human, and include: (a) inhibiting an existing disease-state, i.e., arresting its development or its clinical symptoms; and/or (c) relieving the disease-state, i.e., causing regression of the disease state.

As used herein, “preventing” means causing the clinical symptoms of the disease state not to develop i.e., inhibiting the onset of disease, in a subject that may be exposed to or predisposed to the disease state, but does not yet experience or display symptoms of the disease state.

The following Examples are illustrative and should not be interpreted in any way so as to limit the scope of the invention.

### EXAMPLES

### Example 1

### Synthesis of 3-((S)-1-(Dimethylamino)ethyl)phenyl methyl-((R)-1-phenylpropan-2 yl)carbamate Free Base (3d) and Conjugate Acid Salts (VII)

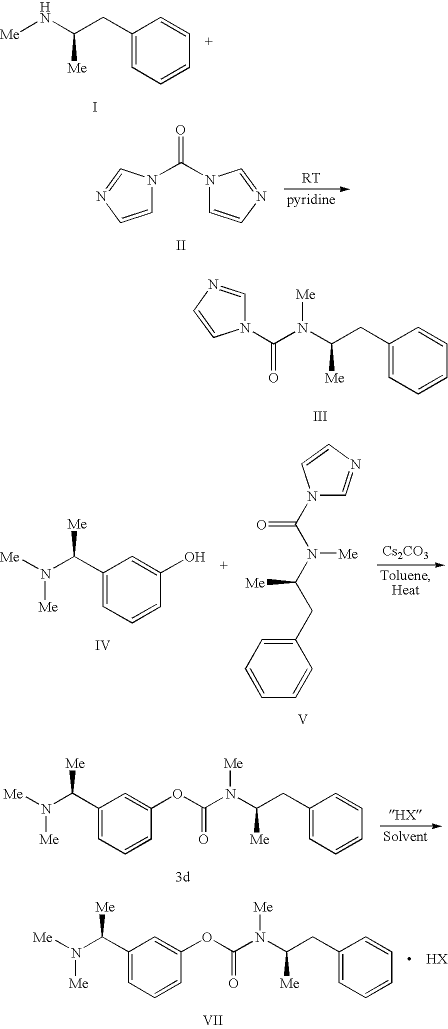

### Example 1A

### Preparation of 3-((S)-1-(Dimethylamino)ethyl)phenyl methyl-((R)-1-phenylpropan-2 yl)carbamate Free Base (3d)

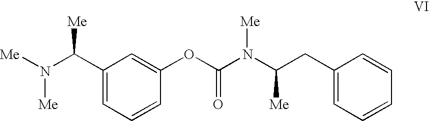

In a 22 L 4-neck flask equipped with addition funnel, mechanical stirrer, “distillation head” with reflux condenser and nitrogen inlet and a thermocouple 638 g (3.86 mol, 1.05 eq.) of (S)-rivaphenol IV and 1791 g (5.5 mol, 1.5 eq.) cesium carbonate were suspended in 9.2 L toluene and subsequently heated to reflux for 30 min. The first distillate (˜120 mL) contained about 1 mL of water and was discarded. Subsequently, 893 g (3.67 mol, 1.0 eq.) activated urea III (Example 1) was dissolved in 3.3 L of toluene (heated to 40° C. to facilitate dissolution) and slowly added to the reaction mixture at a rate that maintained adequate reflux (˜1 h). The addition funnel was rinsed with an additional 100 mL of toluene and this solution was added as well over 10 min. After completed addition the reaction mixture was maintained at reflux overnight. During this time an additional 1 mL of water was collected in the distillation still trap. The reaction mixture was cooled to 90° C. before 1.1 L of 1N sodium hydroxide solution was added. After heating for 3 h to 90° C. The mixture was allowed to cool to rt and transferred into an extraction funnel. The separated toluene layer was washed with 1.6 L of 1N sodium hydroxide before being extracted with aqueous 1N HCL (5.4×1 L+1×2.7 L). The combined acidic extracts were then basified with 10% sodium hydroxide solution to reach pH 12-13 and subsequently extracted with TBME (1×1 L+1×5 L). The combined ether extracts were washed with 11 L 1N sodium hydroxide, two times 1.6 L of water, and 1.1 L of saturated brine before being dried over sodium sulfate. Current chromatographic purity is 99.1% AUC. Solvent evaporation followed by high vacuum drying over weekend afforded 1150 g of the title compound VI (96%).

### Example 1B

### (R)—N-Methyl-N-(1-phenylpropan-2-yl)-1H-imidazole-1-carboxamide (III)

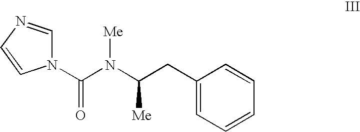

In a 3 L 1-neck flask with mechanical stirring, nitrogen inlet and thermocouple 193.6 g (1.04 mol, 1.0 eq.) L-methamphetamine hydrochloride I was suspended in 1.5 L of pyridine (99+%, 0.1% water maximum) at rt (16° C.). Subsequently, 186.0 g (1.15 mol, 1.1 eq) carbonyldiimidazole (CDI, II) was added in 5 portions over a period of 15 minutes. Upon completion of the addition the temperature had dropped to 12-13° C. The mixture was heated to 90° C. over the period of 1 hour and then kept at this temperature. LCMS control after 1.5 h indicated almost complete conversion with 1.1% starting material remaining. Addition of 8.4 g (0.052 mol, 0.05 eq) CDI and heating to 90° C. for an additional 0.5 h led to complete conversion. The mixture was cooled to 40° C. and stripped of most of its solvent. The resulting suspension was taken into 1.5 L of ethyl acetate and subsequently extracted with three 800 mL portions of water/sat. NaHCO₃ (7/1) to maintain an extraction pH of 7-8. The combined aqueous extracts were extracted with 500 mL of ethyl acetate. LCMS analysis showed ˜10% of the desired product in the second organic layer. Both ethyl acetate phases were combined and dried over magnesium sulfate. The solids were filtered off and washed with little additional ethyl acetate. Evaporation of the solvent under reduced pressure yielded 260 g (>100%) of a yellow solid that contained some pyridine. LCMS analysis confirmed a purity of 99.7%. For purification the yellow solid was suspended in 450 mL of TBME and mechanically stirred for 2 hours to yield a fine white solid in a yellow liquid. Upon addition of 1.5 L of heptane the suspension was further stirred for 16 h before being filtered. Washing with 500 mL of heptane yielded 246 g (97%) of the desired compound III as a fine and slightly yellow solid. LCMS analysis showed a purity of 99.8%. NMR showed 2.3 w/w % of imidazole as impurity.

### Example 1C

### Preparation of 3-((S)-1-(Dimethylamino)ethyl)phenyl methyl-((R)-1-phenylpropan-2 yl)carbamate Hydrogen Fumarate VIIa (HX=Fumaric Acid)

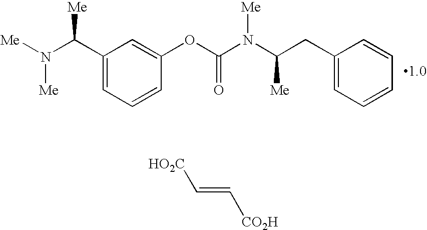

Fumaric acid was dissolved in acetone (5 L), containing 1 kg (1.05 eq) of VI (993 g neat). The resulting solution was diluted with 5 L of isopropyl acetate and mechanically stirred under nitrogen. Massive precipitation was observed overnight. The resulting precipitate was transferred to the Buchner funnels and pressed on the filter funnel to remove 7 L of the mother liquor. The precipitate was washed with 5 L of iPrOAc-acetone 1:1 mixture, pressed on the filter and dried at 30° in vacuum (−25 psi, 50 Torr) with gentle nitrogen flow for four days. The final weight of the title compound 1.2 kg (90% v. free base).

The following data was consistent with the structure shown: ¹H- and ¹³C-NMR were consistent with structure. DSC melting (onset): 101.7° C.; LC/MS analysis indicated an [M+H]=341.1 which is consistent with the molecular weight of the free base portion VI. Elemental Analysis, calculated for C₂₁H₂₈N₂O₂×C₄H₄O₄ (456.53 g/mol): C, 65.77; H, 7.07; N, 6.14. Found: C, 65.61; H, 7.30; N, 5.99. Karl Fischer analysis for moisture indicated 0.18% water.

### Example 1D

### General Procedure for Preparing Salts VII from Compound 3d

Compound VI and the acid (HX) were combined in 10 parts of tetrahydrofuran (2 moles of HX for every 1 mole of VI) and concentrated to dryness. The resulting oil was slurried in 10 parts of isopropylacetate overnight affording a solid precipitate which was isolated by filtration. In some cases, repeating the isopropylacetate slurry treatment was necessary to achieve a consistently melting solid.

### Example 1E

### Preparation of 3-((S)-1-(Dimethylamino)ethyl)phenyl methyl-((R)-1-phenylpropan-2 yl)carbamate Hydrogen Succinate VIIb (HX=Succinic Acid)

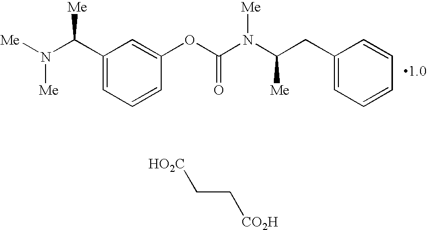

Using the general procedure 2.9 g (78%) of the title compound VIIb was prepared as a white solid.

The following data was consistent with the structure shown: ¹H- and ¹³C-NMR were consistent with structure. DSC melting (onset): 79.0° C.; LC/MS analysis indicated an [M+H]=341.1 which is consistent with the molecular weight of the free base portion VI. Elemental Analysis, calculated for C₂₁H₂₈N₂O₂×C₄H₆O₄ (458.55 g/mol): C, 65.48; H, 7.47; N, 6.11. Found: C, 65.38; H, 7.50; N, 6.08. Karl Fischer analysis for moisture indicated 0.20% water.

### Example 1F

### Preparation of 3-(S)-1-(Dimethylamino)ethyl)phenyl methyl-((R)-1-phenylpropan-2 yl)carbamate Hydrogen Sulfate VIIc (HX=Sulfuric Acid)

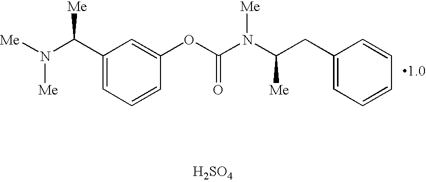

Using the general procedure 7.7 g (87%) of the title compound VIIc was prepared.

The following data was consistent with the structure shown: ¹H- and ¹³C-NMR were consistent with structure. DSC melting (onset): 112.5° C.; LC/MS analysis indicated an [M+H]=341.1 which is consistent with the molecular weight of the free base portion VI. Elemental Analysis, calculated for C₂₁H₂₈N₂O₂×H₂SO₄ (438.54 g/mol): C, 57.51; H, 76.90; N, 6.39; S, 7.31. Found: C, 57.66; H, 7.04; N, 6.31; S, 7.23. Karl Fischer analysis for moisture indicated 0.47% water.

### Example 2

### Synthesis of Cholinesterase Inhibitors

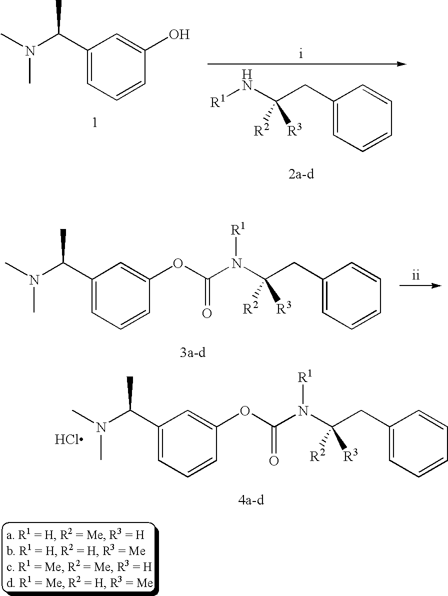

Synthesis of compounds 4a, 4b, 4c, and 4d was accomplished as shown above. Thus, (−)-3-hydroxyphenylethyldimethylamine 1 was reacted with carbonyl diimidazole in dry ethyl acetate to form the activated imidazolide. Addition of the amphetamine isomers (2a-d) gave access to the target carbamates 3a-d in good yields (40-60% after purification by column chromatography). Free base carbamates 3a-d were converted into the corresponding hydrochloride salts 4a-d by addition of hydrogen chloride in diethyl ether followed by removal of the solvents. The identity and purity of the resulting white solids was firmly established by ¹H-NMR and HPLC analysis.

### Example 3

### In Vitro Inhibition of AChE and BuChE

The ability of compounds 4a, 4b, 4c, and 4d to inhibit AChE (human recombinant) and BuChE (human, purified from erythrocytes) was determined. Cholinesterase activity was determined spectrophotometrically by a modified Ellman procedure (Ellman, GL, A new and rapid colorimetric determination of acetylcholinesterase activity. Biochem Pharmacol. 1961, July; 7:88-95). The results of these experiments are provided in the table below and demonstrate that carbamates 4a, 4b, 4c, and 4d were all more potent inhibitors of acetylcholinesterase than the parent structure (rivastigmine).

Carbamates 4a, 4b, 4c, and 4d had less affinity for butyrylcholinesterase than rivastigmine. Consequently, all four inhibitors were more selective AChE inhibitors than rivastigmine. The increase in AChE selectivity was especially pronounced for compounds 4a (incorporating d-amphetamine, which was 225-fold more selective for AChE than rivastigmine) and 4d (incorporating l-methamphetamine, which was 1200-fold more selective than rivastigmine).

Inhibitory concentrations (IC50's) of cholinesterases
4a-d and the parent structure rivastigmine against
recombinant human acetylcholinesterase and purified
human butyrylcholinesterase (plasma).

| | | rhAChE | hBuChE | |
|---|---|---|---|---|
| Compound | Amine | (nM) | (nM) | Selectivity |
| Rivastigmine | N/A | 2615 | 179 | 1:15 |
| 4a | d-amphetamine | 508 | 7322 | 14:1 |
| 4b | l-amphetamine | 404 | 416 | 1:1 |
| 4c | d-methamphetamine | 131 | 334 | 3:1 |
| 4d | l-methamphetamine | 302 | 24400 | 81:1 |

Values represent the mean of two or three independent experiments, each performed in triplicate. Standard deviations were typically within 10% of the IC₅₀ value.

### Example 4

### In Vivo Inhibition of Cholinesterase

The in vivo effectiveness of the stigmine compounds in vivo inhibitors was evaluated. Initially, the Maximum Tolerated Dose (MTD) was determined for each compound. The MTD was defined as the dose at which clear, yet reversible and non-life threatening cholinergic effects were observed. The data in the table below shows that the compounds 4a, 4b, 4c, and 4d were better tolerated than their parent structure rivastigmine.

Inhibition of total brain cholinesterase and total plasma cholinesterase
60 minutes following oral dosing of compounds 4a-d and rivastigmine
at the Maximum Tolerated Dose or highest dose tested.

| | | | | Plasma | Brain |
|---|---|---|---|---|---|
| Compound | Amine | MTD (mg/kg) | | ChEI | ChEI |
| Rivastigmine^{#} | N/A | 5 | mg/kg | 39% | 56% |
| 4a | d-amphetamine | >100 | mg/kg | 59% | 72% |
| 4b | l-amphetamine | 32 | mg/kg | 26% | 25% |
| 4c | d-methamphetamine | >64 | mg/kg | 50% | 50% |
| 4d | l-methamphetamine | 10 | mg/kg | 45% | 64% |

Values represent the mean of n = 4 animals per group. Standard deviations were typically within 10% of the determined cholinesterase activity.
^{#}Rivastigmine data is the mean of three measurements between 30 and 180 min following dosing.

To establish the in vivo cholinesterase inhibition properties of the compounds, rats were dosed orally with saline solutions of the test compounds at the MTD (or at the highest dose tested in case of 4a and 4e). Following administration, rats were sacrificed and blood and brain samples were collected, processed and ChE activity was determined spectrophotometrically as described above. Initially, cholinesterase inhibition was quantified at 30, 60 and 180 min following oral dosing of rivastigmine to assess time-dependence. As there was no consistent difference in the level of inhibition achieved between these times (Bonferroni t-test), which is in accord with reports of its long duration of pseudo-irreversible inhibition, plasma and brain levels of cholinesterase inhibition of all other compounds were determined at a single time point (60 min). All compounds resulted in significant inhibition of cholinesterase following oral dosing in both plasma and brain, demonstrating the oral bioavailability and blood-brain barrier penetration of compounds 4a-d.

### Example 5

### Amphetamine Release

Next, the release of amphetamine from inhibited cholinesterase was investigated indirectly by monitoring reconstitution of enzyme activity as described above. Recombinant human cholinesterase was incubated with an excess of carbamates 4a-d, resulting in >80% inhibition of enzyme activity. The enzyme was separated from small organic molecules (carbamate and possible degradation products) by size exclusion chromatography over a Sephadex column. Purified, inhibited enzyme was incubated in phosphate buffer at 37° C., aliquots were drawn at various time points and the enzymatic activity was determined as described above. The data were plotted as the natural logarithm of the percent inhibition versus time according to a first-order kinetics model. The decarbamylation rate constant k was determined as the slope of this line. The halflife t_{1/2} is directly related to k (k=−ln(0.500)/t_{1/2}).

The results of the decarbamylation experiments are presented in the table below.

Decarbamylation rates (k) and decarbamylation half-lives (t_{1/2})
following inhibition of rhAChE by compounds 4a-d and rivastigmine

| | |
|---|---|
| k (h⁻¹) | t_{1/2} |

| | | |
|---|---|---|
| Compound | Amine | (h) |

| | | | |
|---|---|---|---|
| Rivastigmine | N/A | 0.007 | >24 |
| 4a | d-amphetamine | 0.558 | 1.2 |
| 4b | l-amphetamine | 0.057 | 12 |
| 4c | d-methamphetamine | 0.003 | >24 |
| 4d | l-methamphetamine | 0.011 | >24 |

In order to unequivocally demonstrate that enzyme reconstitution results in release of amphetamine, aliquots from the decarbamylation experiments with 4a were analyzed for levels of amphetamine using an LC/MS/MS method. It was demonstrated that aliquots drawn at t=0 did not contain amphetamine, whereas aliquots drawn 4 h later (corresponding to 80% reconstitution) contained 53 ng/mL of d-amphetamine. Similarly, plasma collected from rats one hour following oral doses (64 mg/kg) of 4a contained significant amounts of amphetamine (17 ng/mL).

### Example 6

### Memory Enhancement

To determine whether compound 4a had any memory enhancing properties, its effects were tested in a scopolamine model of passive avoidance (C. Bejar, R.-H. Wang, M. Weinstock, “Effects of rivastigmine on scopolamine-induced memory impairment in rats,”, Eur. J. Pharmacol. 383, 1999, 231-240). Prior to training, rats were injected with scopolamine hydrobromide (0.2 mg/kg, s.c, 40 minutes before training) and with either saline or compound 4a (8, 12, 16 or 24 mg/kg, i.p, 30 minutes prior to training). The animals received a retention test 24 hours following training. The retention test was identical to training except that no foot-shock or drug was delivered. Latency to enter the dark chamber was recorded.

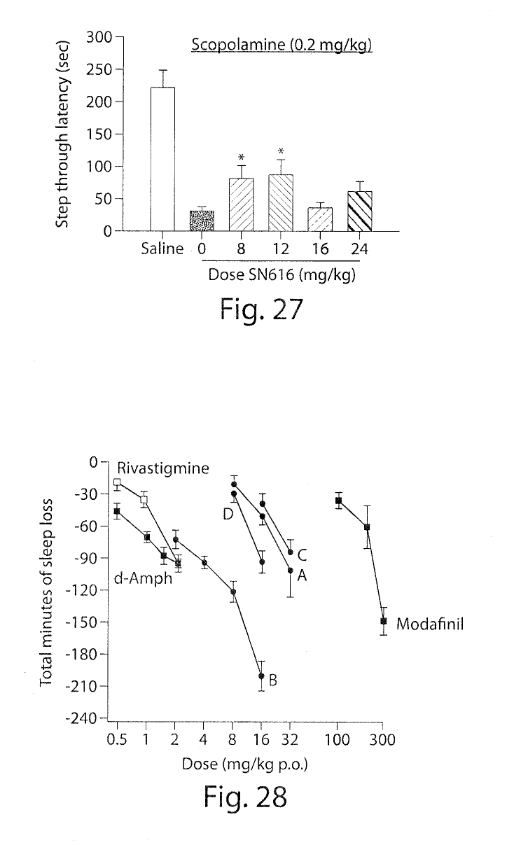

Results (FIG. 27) demonstrated that pre-training administration of scopolamine produced a robust and statistically significant amnesia (p<0.0001). This amnesia could be alleviated by doses of 8 and 12 mg/kg of compound 4a (p<0.01). These results clearly demonstrate that compound 4a is able to improve mnemonic performance in rats.

### Example 7

### Formulation Summary

Formulation studies indicate the compound 3d has favorable properties for development as an oral drug. Solid compound 3d is stable to heat (40° C.) for 7 days, to UV for 220 W-h/m², and no change after visible light exposure of 7.3×10 h equivalent. Compound 3d solutions are stable at pH 4, 5, 6, after 7 days based on HPLC recovery experiments. Compound 3d is highly soluble (>50 mg/mL) at pH 3-6. Stability indicating HPLC method qualified for use in formulation work (linearity, precision, accuracy, recovery).

Polymorph studies indicate that compound 3d is highly crystalline and one form is predominant. Hygroscopicity testing at 40° C./75 RH indicated stability over 7 days by differential scanning calorimetry (DSC) and X-ray powder diffraction (XRPD) studies. Mechanical stress ball pine indicates instability to extreme thermal conditions (compound “gums”), likely due to compound 3d melting point which is ca. 110° C. Sixteen vehicles for oral dosing (rodents) were tested and all behave equally well (>50 mg/mL). Water, HMC, or HPMC (0.25, 0.5%) are the preferred choices.

### Example 8

### Salt Selection

Batches of compound 3d were prepared as the free base. Because the synthesis of compound 3d free base resulted in an oil, which is not practical for development, focus shifted to identifying a suitable a salt form. Fifteen salts of compound 3d were initially screened. The initial salt selection screen resulted in oils and gums. A second salt selection screen was conducted over a 3 month period to screen 40 acids. The second salt selection screen resulted in only gums/oils. Crystallization studies were then conducted using “best solvents” not yet tried. The initial crystallization study was repeated using the newly selected solvents and avoiding heat due to the low melting point of compound 3d salts. Several solvents/conditions produced solids; isopropyl acetate (IPA) was selected for further study. The top 10 preferred salt forms were re-screened using IPA solvent. Three solid forms of compound 3d were selected for further study. The 3 solid forms chosen were succinate, fumarate, and sulfate salt (1:1 ratio). The 3 solid forms were screened for “druggable” properties: melting point, crystallity, solubility, hygroscopicity, stability, etc. The hydrogen succinate salt of compound 3d afforded a low melting point (60-70° C.) and amorphous character was shown by microscopy and XRPD.

Further studies were conducted on the hydrogen fumarate (mp 110° C.) and hydrogen sulfate (mp 120° C.) salts of compound 3d were conducted.

### Example 9

### Peak List for X-Ray Diffractogram of Hydrogen Fumarate Salt Shown in FIG. 16

| 2-Theta | d(Å) | BG | Height | H % | Area | A % | FWHM |
|---|---|---|---|---|---|---|---|
| 4.221 | 20.9171 | 377 | 173 | 6.7 | 3363 | 5.1 | 0.165 |
| 8.451 | 10.4544 | 290 | 107 | 4.1 | 2422 | 3.7 | 0.193 |
| 9.510 | 9.2920 | 316 | 96 | 3.7 | 5616 | 8.5 | 0.498 |
| 9.759 | 9.0555 | 329 | 148 | 5.7 | 5616 | 8.5 | 0.322 |
| 10.670 | 8.2846 | 336 | 225 | 8.7 | 8415 | 12.8 | 0.317 |
| 11.919 | 7.4192 | 315 | 162 | 6.3 | 7377 | 11.2 | 0.387 |
| 13.891 | 6.3701 | 340 | 236 | 9.2 | 5509 | 8.4 | 0.198 |
| 15.319 | 5.7792 | 332 | 418 | 16.2 | 16246 | 24.7 | 0.331 |
| 16.641 | 5.3229 | 396 | 311 | 12.0 | 13291 | 20.2 | 0.364 |
| 17.010 | 5.2084 | 367 | 1712 | 66.4 | 63377 | 96.2 | 0.315 |
| 17.648 | 5.0215 | 478 | 89 | 3.5 | 1412 | 2.1 | 0.135 |
| 18.401 | 4.8176 | 548 | 218 | 8.5 | 5489 | 8.3 | 0.214 |
| 18.800 | 4.7163 | 533 | 1849 | 71.6 | 62424 | 94.8 | 0.287 |
| 19.431 | 4.5646 | 703 | 217 | 8.4 | 2718 | 4.1 | 0.107 |
| 20.412 | 4.3473 | 615 | 204 | 7.9 | 9152 | 13.9 | 0.381 |
| 20.641 | 4.2996 | 591 | 184 | 7.1 | 9152 | 13.9 | 0.424 |
| 21.340 | 4.1603 | 515 | 2581 | 100.0 | 65870 | 100.0 | 0.217 |
| 23.280 | 3.8178 | 438 | 541 | 21.0 | 22089 | 33.5 | 0.347 |
| 23.600 | 3.7668 | 456 | 635 | 24.6 | 24683 | 37.5 | 0.330 |
| 24.618 | 3.6133 | 460 | 88 | 3.4 | 3950 | 6.0 | 0.382 |
| 25.001 | 3.5588 | 424 | 119 | 4.6 | 3950 | 6.0 | 0.281 |
| 25.720 | 3.4610 | 425 | 1711 | 66.3 | 40559 | 61.6 | 0.201 |
| 26.421 | 3.3707 | 421 | 313 | 12.1 | 11227 | 17.0 | 0.305 |
| 27.801 | 3.2065 | 389 | 121 | 4.7 | 3260 | 4.9 | 0.229 |
| 28.340 | 3.1467 | 379 | 181 | 7.0 | 7421 | 11.3 | 0.348 |
| 28.681 | 3.1100 | 370 | 96 | 3.7 | 6372 | 9.7 | 0.563 |
| 30.129 | 2.9638 | 351 | 472 | 18.3 | 14090 | 21.4 | 0.254 |
| 30.729 | 2.9072 | 369 | 120 | 4.6 | 5042 | 7.7 | 0.358 |
| 31.550 | 2.8334 | 378 | 177 | 6.9 | 4838 | 7.3 | 0.233 |
| 32.148 | 2.7820 | 360 | 71 | 2.8 | 3089 | 4.7 | 0.370 |
| 32.500 | 2.7528 | 353 | 61 | 2.4 | 1500 | 2.3 | 0.208 |
| 34.156 | 2.6230 | 295 | 65 | 2.5 | 3724 | 5.7 | 0.487 |
| 34.480 | 2.5990 | 292 | 75 | 2.9 | 4629 | 7.0 | 0.522 |
| 36.500 | 2.4597 | 294 | 65 | 2.5 | 2600 | 3.9 | 0.338 |
| 37.809 | 2.3775 | 265 | 55 | 2.1 | 762 | 1.2 | 0.119 |
| 40.755 | 2.2122 | 273 | 62 | 2.4 | 847 | 1.3 | 0.117 |
| 41.930 | 2.1529 | 287 | 92 | 3.6 | 2322 | 3.5 | 0.214 |
| 43.560 | 2.0760 | 270 | 289 | 11.2 | 8615 | 13.1 | 0.253 |
| 45.590 | 1.9882 | 262 | 65 | 2.5 | 2741 | 4.2 | 0.356 |
| 48.057 | 1.8917 | 245 | 55 | 2.1 | 2607 | 4.0 | 0.400 |

### Example 10

### Peak List for X-Ray Diffractogram of Hydrogen Succinate Salt Shown in FIG. 18

| 2-Theta | d(Å) | BG | Height | H % | Area | A % | FWHM |
|---|---|---|---|---|---|---|---|
| 4.190 | 21.0716 | 382 | 176 | 6.3 | 3738 | 3.5 | 0.181 |
| 8.430 | 10.4798 | 295 | 159 | 5.7 | 2712 | 2.6 | 0.145 |
| 10.360 | 8.5319 | 315 | 660 | 23.6 | 17735 | 16.8 | 0.229 |
| 11.410 | 7.7491 | 309 | 728 | 26.1 | 21540 | 20.4 | 0.251 |
| 13.600 | 6.5055 | 301 | 406 | 14.6 | 10266 | 9.7 | 0.215 |
| 14.071 | 6.2890 | 311 | 265 | 9.5 | 6278 | 5.9 | 0.202 |
| 14.900 | 5.9409 | 314 | 677 | 24.3 | 17805 | 16.9 | 0.223 |
| 15.860 | 5.5832 | 341 | 312 | 11.2 | 7850 | 7.4 | 0.214 |
| 16.620 | 5.3297 | 410 | 1072 | 38.5 | 42931 | 40.7 | 0.340 |
| 16.940 | 5.2296 | 389 | 1467 | 52.6 | 59059 | 56.0 | 0.342 |
| 17.650 | 5.0209 | 437 | 423 | 15.2 | 10329 | 9.8 | 0.208 |
| 18.550 | 4.7794 | 549 | 1023 | 36.7 | 21179 | 20.1 | 0.176 |
| 19.130 | 4.6357 | 437 | 2614 | 93.7 | 76323 | 72.3 | 0.248 |
| 19.470 | 4.5556 | 538 | 365 | 13.1 | 11293 | 10.7 | 0.263 |
| 20.149 | 4.4034 | 427 | 272 | 9.7 | 5527 | 5.2 | 0.173 |
| 20.830 | 4.2610 | 449 | 578 | 20.7 | 37689 | 35.7 | 0.554 |
| 21.260 | 4.1758 | 431 | 2789 | 100.0 | 105518 | 100.0 | 0.322 |
| 22.299 | 3.9834 | 429 | 235 | 8.4 | 5616 | 5.3 | 0.203 |
| 22.921 | 3.8769 | 489 | 265 | 9.5 | 5024 | 4.8 | 0.161 |
| 23.450 | 3.7906 | 421 | 670 | 24.0 | 58099 | 55.1 | 0.737 |
| 23.770 | 3.7403 | 450 | 1564 | 56.1 | 65703 | 62.3 | 0.357 |
| 24.280 | 3.6628 | 497 | 247 | 8.8 | 3483 | 3.3 | 0.120 |
| 25.560 | 3.4822 | 373 | 1635 | 58.6 | 68084 | 64.5 | 0.354 |
| 26.720 | 3.3336 | 354 | 344 | 12.3 | 11191 | 10.6 | 0.277 |
| 27.171 | 3.2793 | 340 | 263 | 9.4 | 14768 | 14.0 | 0.478 |
| 29.010 | 3.0755 | 321 | 934 | 33.5 | 33075 | 31.3 | 0.301 |
| 29.950 | 2.9810 | 348 | 256 | 9.2 | 6077 | 5.8 | 0.202 |
| 30.751 | 2.9052 | 353 | 94 | 3.4 | 3799 | 3.6 | 0.343 |
| 31.554 | 2.8331 | 371 | 106 | 3.8 | 1039 | 1.0 | 0.084 |
| 31.910 | 2.8023 | 322 | 294 | 10.5 | 10740 | 10.2 | 0.311 |
| 32.530 | 2.7503 | 353 | 527 | 18.9 | 15029 | 14.2 | 0.242 |
| 33.420 | 2.6790 | 327 | 131 | 4.7 | 5632 | 5.3 | 0.365 |
| 34.440 | 2.6020 | 316 | 108 | 3.9 | 3306 | 3.1 | 0.261 |
| 34.841 | 2.5729 | 300 | 114 | 4.1 | 3254 | 3.1 | 0.243 |
| 35.990 | 2.4934 | 282 | 58 | 2.1 | 1203 | 1.1 | 0.176 |
| 36.651 | 2.4499 | 266 | 105 | 3.8 | 6259 | 5.9 | 0.504 |
| 37.429 | 2.4008 | 267 | 116 | 4.2 | 3220 | 3.1 | 0.235 |
| 38.287 | 2.3489 | 267 | 75 | 2.7 | 2749 | 2.6 | 0.313 |
| 38.750 | 2.3219 | 257 | 57 | 2.0 | 3288 | 3.1 | 0.490 |
| 39.941 | 2.2554 | 256 | 212 | 7.6 | 10513 | 10.0 | 0.421 |
| 41.311 | 2.1837 | 279 | 176 | 6.3 | 6075 | 5.8 | 0.293 |
| 42.081 | 2.1455 | 297 | 99 | 3.5 | 1798 | 1.7 | 0.155 |
| 42.754 | 2.1133 | 304 | 58 | 2.1 | 958 | 0.9 | 0.141 |
| 43.359 | 2.0852 | 296 | 239 | 8.6 | 13269 | 12.6 | 0.471 |
| 44.501 | 2.0343 | 270 | 64 | 2.3 | 1649 | 1.6 | 0.220 |
| 46.019 | 1.9706 | 264 | 123 | 4.4 | 5328 | 5.0 | 0.369 |
| 46.812 | 1.9391 | 268 | 181 | 6.5 | 5336 | 5.1 | 0.251 |
| 47.833 | 1.9001 | 266 | 117 | 4.2 | 3651 | 3.5 | 0.266 |
| 49.130 | 1.8529 | 255 | 120 | 4.3 | 4282 | 4.1 | 0.302 |

### Example 11

### Peak List for X-Ray Diffractogram of Hydrogen Sulfate Salt Shown in FIG. 20

| 2-Theta | d(Å) | BG | Height | H % | Area | A % | FWHM |
|---|---|---|---|---|---|---|---|
| 3.340 | 26.4331 | 533 | 3038 | 100.0 | 57856 | 90.0 | 0.162 |
| 6.667 | 13.2476 | 323 | 79 | 2.6 | 3559 | 5.5 | 0.384 |
| 10.061 | 8.7848 | 302 | 1035 | 34.0 | 20333 | 31.6 | 0.167 |
| 10.601 | 8.3388 | 291 | 342 | 11.3 | 7390 | 11.5 | 0.183 |
| 13.449 | 6.5782 | 340 | 611 | 20.1 | 12589 | 19.6 | 0.175 |
| 13.949 | 6.3435 | 382 | 1409 | 46.4 | 28394 | 44.2 | 0.171 |
| 14.370 | 6.1588 | 398 | 347 | 11.4 | 4731 | 7.4 | 0.116 |
| 14.921 | 5.9325 | 398 | 983 | 32.4 | 21041 | 32.7 | 0.182 |
| 15.340 | 5.7715 | 355 | 392 | 12.9 | 17597 | 27.4 | 0.382 |
| 15.770 | 5.6149 | 369 | 688 | 22.6 | 13593 | 21.1 | 0.168 |
| 16.391 | 5.4036 | 317 | 220 | 7.2 | 4587 | 7.1 | 0.177 |
| 16.869 | 5.2515 | 328 | 419 | 13.8 | 10637 | 16.5 | 0.216 |
| 17.160 | 5.1632 | 335 | 517 | 17.0 | 10469 | 16.3 | 0.172 |
| 17.540 | 5.0521 | 336 | 190 | 6.2 | 3438 | 5.3 | 0.154 |
| 18.060 | 4.9079 | 329 | 1825 | 60.1 | 38019 | 59.1 | 0.177 |
| 19.070 | 4.6502 | 423 | 459 | 15.1 | 13138 | 20.4 | 0.243 |
| 19.420 | 4.5671 | 465 | 2241 | 73.7 | 45484 | 70.8 | 0.173 |
| 19.930 | 4.4513 | 467 | 690 | 22.7 | 64278 | 100.0 | 0.792 |
| 20.230 | 4.3861 | 420 | 1698 | 55.9 | 62843 | 97.8 | 0.315 |
| 20.581 | 4.3121 | 428 | 149 | 4.9 | 1938 | 3.0 | 0.110 |
| 21.340 | 4.1603 | 329 | 1186 | 39.0 | 24848 | 38.7 | 0.178 |
| 22.210 | 3.9993 | 324 | 496 | 16.3 | 12758 | 19.8 | 0.219 |
| 22.700 | 3.9141 | 313 | 414 | 13.6 | 13033 | 20.3 | 0.267 |
| 23.851 | 3.7278 | 338 | 686 | 22.6 | 24796 | 38.6 | 0.307 |
| 24.150 | 3.6822 | 311 | 1003 | 33.0 | 52384 | 81.5 | 0.444 |
| 24.780 | 3.5901 | 348 | 372 | 12.3 | 7261 | 11.3 | 0.166 |
| 25.261 | 3.5228 | 341 | 258 | 8.5 | 4499 | 7.0 | 0.148 |
| 25.910 | 3.4359 | 321 | 500 | 16.5 | 27955 | 43.5 | 0.475 |
| 26.180 | 3.4011 | 341 | 693 | 22.8 | 20309 | 31.6 | 0.249 |
| 26.680 | 3.3385 | 329 | 458 | 15.1 | 8779 | 13.7 | 0.163 |
| 27.108 | 3.2868 | 375 | 142 | 4.7 | 1459 | 2.3 | 0.087 |
| 27.449 | 3.2467 | 314 | 452 | 14.9 | 16979 | 26.4 | 0.319 |
| 27.941 | 3.1907 | 303 | 313 | 10.3 | 17275 | 26.9 | 0.470 |
| 29.000 | 3.0765 | 309 | 187 | 6.2 | 3257 | 5.1 | 0.148 |
| 29.710 | 3.0046 | 304 | 226 | 7.4 | 8056 | 12.5 | 0.304 |
| 30.310 | 2.9465 | 286 | 326 | 10.7 | 13522 | 21.0 | 0.352 |
| 31.329 | 2.8529 | 256 | 79 | 2.6 | 2731 | 4.2 | 0.293 |
| 31.929 | 2.8006 | 250 | 282 | 9.3 | 5909 | 9.2 | 0.178 |
| 32.339 | 2.7661 | 253 | 68 | 2.2 | 1413 | 2.2 | 0.177 |
| 33.229 | 2.6940 | 269 | 155 | 5.1 | 5331 | 8.3 | 0.292 |
| 33.719 | 2.6560 | 258 | 127 | 4.2 | 7429 | 11.6 | 0.498 |
| 34.028 | 2.6325 | 274 | 100 | 3.3 | 3828 | 6.0 | 0.326 |
| 34.690 | 2.5838 | 257 | 310 | 10.2 | 8925 | 13.9 | 0.245 |
| 35.131 | 2.5524 | 254 | 159 | 5.2 | 3510 | 5.5 | 0.188 |
| 35.690 | 2.5137 | 259 | 222 | 7.3 | 6324 | 9.8 | 0.242 |
| 36.589 | 2.4539 | 270 | 107 | 3.5 | 3955 | 6.2 | 0.315 |
| 37.141 | 2.4188 | 277 | 58 | 1.9 | 3122 | 4.9 | 0.461 |
| 37.699 | 2.3842 | 255 | 182 | 6.0 | 5853 | 9.1 | 0.273 |
| 38.180 | 2.3553 | 244 | 106 | 3.5 | 3012 | 4.7 | 0.243 |
| 39.009 | 2.3071 | 257 | 81 | 2.7 | 1363 | 2.1 | 0.143 |
| 39.591 | 2.2745 | 244 | 90 | 3.0 | 3399 | 5.3 | 0.320 |
| 39.959 | 2.2544 | 259 | 162 | 5.3 | 4004 | 6.2 | 0.210 |
| 40.659 | 2.2172 | 245 | 84 | 2.8 | 1384 | 2.2 | 0.140 |
| 41.843 | 2.1572 | 252 | 69 | 2.3 | 2798 | 4.4 | 0.346 |
| 42.409 | 2.1297 | 259 | 129 | 4.2 | 4085 | 6.4 | 0.269 |
| 42.698 | 2.1159 | 263 | 80 | 2.6 | 2704 | 4.2 | 0.286 |
| 43.489 | 2.0792 | 259 | 174 | 5.7 | 6524 | 10.1 | 0.318 |
| 44.071 | 2.0531 | 253 | 125 | 4.1 | 3242 | 5.0 | 0.220 |
| 45.300 | 2.0002 | 260 | 66 | 2.2 | 1594 | 2.5 | 0.206 |
| 45.681 | 1.9844 | 244 | 75 | 2.5 | 6748 | 10.5 | 0.767 |
| 46.089 | 1.9678 | 250 | 66 | 2.2 | 2966 | 4.6 | 0.379 |
| 47.149 | 1.9260 | 234 | 89 | 2.9 | 3751 | 5.8 | 0.357 |
| 48.079 | 1.8909 | 243 | 67 | 2.2 | 757 | 1.2 | 0.095 |
| 48.941 | 1.8596 | 258 | 101 | 3.3 | 2501 | 3.9 | 0.210 |

### Example 12

### Wake Promotion

Certain clinical conditions are characterized by unpredictable bouts of sleepiness that can interfere with the ability to conduct activities of daily living, such as driving. Examples are narcolepsy, and disturbances of diurnal rhythm, such as adjustment to shift work. Currently approved therapies for such conditions are amphetamines and modifenil. Significant limitations of available therapies include rebound hypersomnolence and abuse potential.

Test compounds in various dose ranges or vehicle were administered to male Wistar rats 5 hours after lights on (CT-5). EEG, EMG, locomotor activity, drink- and food-related activity, and body temperature were concurrently monitored for 30 hr before and after treatment from rats living in separate isolated recording chambers. Sleep-wake discriminations were carried out using SCORE2004™, proprietary real-time hardware and software technology of Hypnion, Inc. Comparisons were made between the reference compounds: d-amphetamine, rivastigmine, modafinil, and the following test compounds:

| Compound # | Compound Name/Letter Code |
|---|---|
| 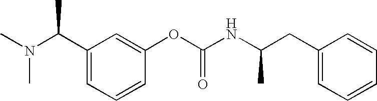 | s-riva-l-amphetamine/A |
| 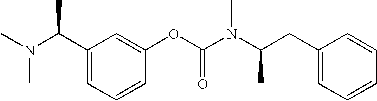 | s-riva-l-methamphetamine/B |
| 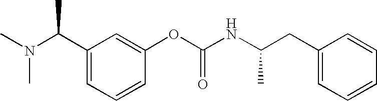 | s-riva-d-amphetamine/C |
| 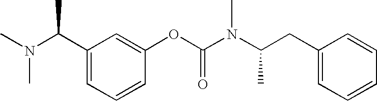 | s-riva-d-methamphetamine/D |

Administration of d-amphetamine or modafinil increased the duration of wakefulness (i.e., increased the total number of minutes of sleep loss) in a dose-dependent manner. Although not approved as a wake promoting agent, rivastigmine also increased wakefulness. Higher doses of reference compounds were not tested because of tolerability. Similarly, Compounds A, B, C and D caused a dose-related increase in wakefulness. Of these, Compound B caused an unexpectedly long increase in wakefulness that surpassed that seen with the reference compounds tested (See, FIG. 28).

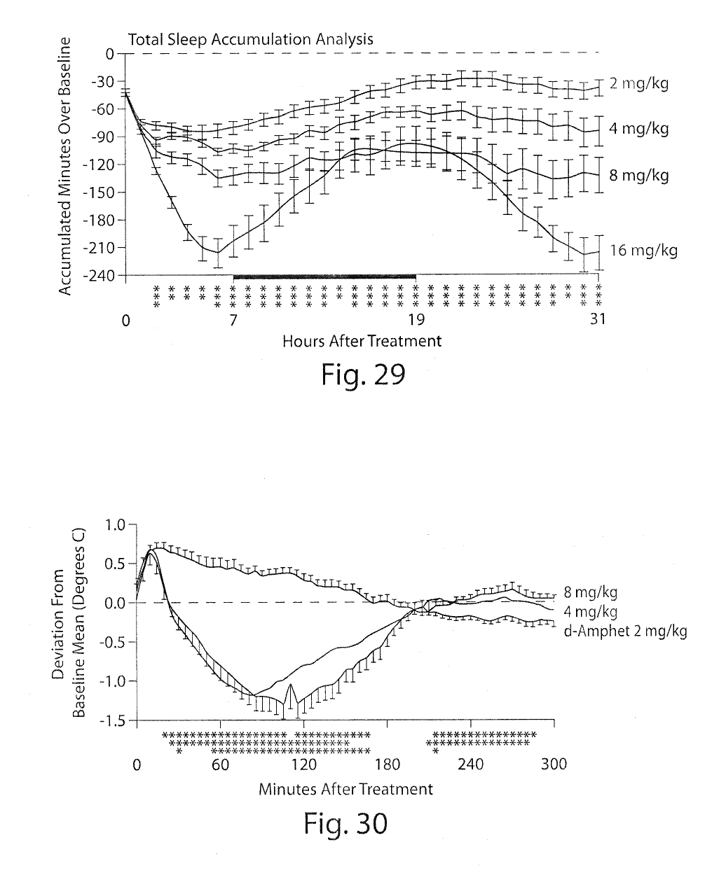

Unlike rivastigmine, rebound hypersomnolence was not observed following administration of Compound B (See, FIG. 29). This is an unexpected finding that would not be predicted from the known actions of the compound's component stigmine or amine.

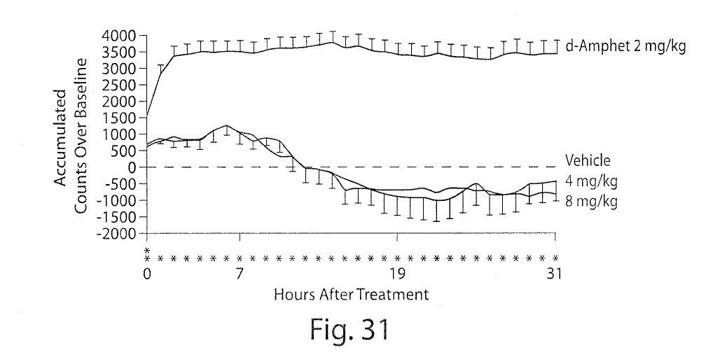

Compound B, also differed in other unexpected ways from the reference compounds with respect to its effects on body temperature and locomotor activity. Unlike d-amphetamine, Compound B did not cause an increase in body temperature (hyperthermia), but rather caused an opposite reduction in body temperature (hypothermia; See, FIG. 30). Moreover, unlike d-methamphetamine, Compound B did not cause locomotor hyperactivity, indicating an absence of stimulant activity (See, FIG. 31). Further evidence for a lack of psychostimulant activity in Compound B is given in Example 13.

### Example 13

### Lack of Psychostimulant-Like Effect

Drug discrimination is an operant paradigm that enables assessment of drug abuse liability (Yasar & Bergman, 1994. Amphetamine-like effect of 1-deprenyl (selegiline) in drug discrimination studies. Clin. Pharmacol. Therap. 56 (S78), 768-773). In this paradigm, psychostimulant properties of compounds may be determined in rats trained to discriminate methamphetamine from saline. Hungry rats are initially placed in a test apparatus where they learn that pressing either of two levers results in delivery of a food pellet. Once lever pressing has been established, rats learn that if they are pretreated with methamphetamine, they must now choose (for example) the left hand lever in order to obtain food. On other days, rats are pretreated with vehicle, and must then select the opposite lever in order to obtain food. In this way, rats must learn to use the interoceptive cues generated by the psychostimulant drug to guide its choice of levers. Once lever pressing to a predetermined criterion has been established, a test compound can be administered. On these days, pressing either lever results in food, permitting examination of whether the rats select the methamphetamine or the saline lever. If the rat chooses the methamphetamine lever, the test drug is said to have shown stimulus generalization; that is, it is perceived to be methamphetamine-like by the rat. After administration of Compound B (0.1-3.2 mg/kg i.p. or 0.32-10 mg/kg p.o.), rats did not select the methamphetamine lever, indicating a lack of stimulus generalization. These findings suggest that Compound B may possess wake promoting activity without psychostimulant drug abuse liability.

### EQUIVALENTS

While this invention has been particularly shown and described with references to preferred embodiments thereof, it will be understood by those skilled in the art that various changes in form and details may be made therein without departing from the scope of the invention encompassed by the appended claims.

What is claimed:

1. A hydrogen fumarate salt of compound 3d: 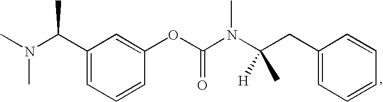
characterized by an x-ray diffraction pattern substantially similar to that set forth in FIG. 16.

2. A pharmaceutical composition comprising the hydrogen fumarate salt of compound 3d: 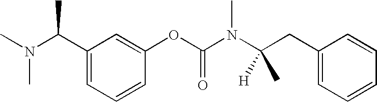
and a pharmaceutically acceptable carrier or excipient.

3. The composition according to claim 2, wherein the compound has a purity of greater than 98.0% as determined by LCMS.

4. The composition according to claim 2, wherein the compound contains less than 2% impurity.

5. The composition according to claim 2, wherein the compound contains less than 2% d-methamphetamine.

6. A method of treating or preventing a nervous system condition, cholinergic deficiency or glaucoma in an individual by administering a composition according to claim 2.

7. The method of claim 6, wherein said nervous system condition is selected from a central nervous system condition, a peripheral nervous system condition, and autonomic nervous system condition.

8. The method of claim 6, wherein said central nervous system condition is selected from Parkinson's disease, memory impairment, and cognitive impairment.

9. The method of claim 8, wherein said memory impairment is selected from Alzheimer's disease, age-associated memory loss, impairment in memory consolidation, impairment in short term memory, mild cognitive impairment, and multiple sclerosis.

10. A method of treating or preventing a condition associated with acetylcholinesterase activity in an individual by administering a composition according to claim 2, wherein the condition is selected from delayed gastric emptying, attention deficit hyperactivity disorder (ADHD), phobia, stroke, multiple sclerosis, sleep disorder, psychiatric disorder, pain, anticholinergic drug overdose, tobacco dependence, and spasticity.

11. A method of promoting wakefulness in an individual by administering a composition according to claim 2.

12. The method of claim 11, wherein the individual suffers from a disorder or condition selected from wakefulness disorders, hypersomnia, sleep apnea, sleep disorders of central origin, fatigue, excessive daytime sleepiness associated with narcolepsy, fatigue and excessive sleepiness associated with a depressive disorder or with antidepressant therapy.

13. A process for preparing 3-((S)-1-(dimethylamino)ethyl)phenyl methyl-((R)-1-phenylpropan-2 yl)carbamate hydrogen fumarate comprising the steps of:
(1) reacting L-methamphetamine with carbonyldiimidazole to form (R)—N-Methyl-N-(1-phenylpropan-2-yl)-1H-imidazole-1-carboxamide;
(2) reacting (R)—N-Methyl-N-(1-phenylpropan-2-yl)-1H-imidazole-1-carboxamide with (S)-rivalphenol to form 3-((S)-1-(dimethylamino)ethyl)phenyl methyl-((R)-1-phenylpropan-2 yl)carbamate; and
(3) contacting 3-((S)-1-(dimethylamino)ethyl)phenyl methyl-((R)-1-phenylpropan-2 yl)carbamate with fumaric acid to yield 3-((S)-1-(dimethylamino)ethyl)phenyl methyl-((R)-1-phenylpropan-2 yl)carbamate hydrogen fumarate.

14. A compound having the structure 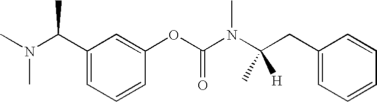
or a pharmaceutically acceptable salt thereof.
